(19) United States
(12) Patent Application Publication
(10) Pub. No.: US 2008/0311039 A1
(43) Pub. Date: Dec. 18, 2008

(54) Therapeutic and Prognostic Factor Yy1 in Human Cancer

(75) Inventors: Benjamin Bonavida, Los Angeles, CA (US); Lee Goodglick, Los Angeles, CA (US); Hermes Gaban, Los Angeles, CA (US); Stephan Horvath, Los Angeles, CA (US); David Seligson, Los Angeles, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 11/662,220

(22) PCT Filed: Sep. 9, 2005
(86) PCT No.: PCT/US05/32391

§ 371 Date: Sep. 8, 2008

Related U.S. Application Data
(60) Provisional application No. 60/608,829, filed on Sep. 9, 2004.
(60) Provisional application No. 60/658,561, filed on Mar. 3, 2005.

(51) Int. Cl. A61K 49/00; C07H 21/04; A61P 35/00; A61K 33/00; A61K 31/7105; G01N 33/574; C12Q 1/68; C12Q 1/02

(52) U.S. Cl. 424/9.1; 536/24.33; 424/718; 514/44; 435/7.23; 435/6; 435/29

(57) ABSTRACT

The present invention provides for the first time YY1, a transcription factor gene over-expressed and/or functionally overactive in human cancer. The present invention provides methods of diagnosing and providing a prognosis for cancer such as prostate cancer, as well as methods of drug discovery. YY1 is also a therapeutic target for treatment of cancer resistant to conventional and experimental cancer therapeutics. Inhibition of YY1 expression and/or activity sensitizes resistant tumor cells to cytotoxic treatments, including chemotherapy, radiation therapy, hormonal therapy, and immunotherapy.

### CROSS-REFERENCES TO RELATED APPLICATIONS

This application claims the benefit of U.S. Provisional Patent Application No. 60/608,829, filed Sep. 9, 2004, and U.S. Provisional Patent Application No. 60/658,561, filed Mar. 3, 2005, the contents of each of which is hereby incorporated herein by reference in its entirety for all purposes.

### STATEMENT AS TO RIGHTS TO INVENTIONS MADE UNDER FEDERALLY SPONSORED RESEARCH OR DEVELOPMENT

This invention was made with Government support under National Cancer Institute Grant No. CA-86366 and Department of Defense/US Army Grant DAMD 17-02-1-0023. The Government has certain rights in this invention.

### BACKGROUND OF THE INVENTION

Cancer is the second leading cause of death behind heart disease. Cancer incidence and death figures account for about 10% of the U.S. population in certain areas of the United States (National Cancer Institute's Surveillance, Epidemiology, and End Results (SEER) database and Bureau of the Census statistics; see, Harrison's Principles of Internal Medicine, Kasper, et al., 16^{th} ed., 2005, Chapter 66). The five leading causes of cancer deaths among men are lung cancer, prostate cancer, colon and rectum cancer, pancreatic cancer and leukemias. The five leading causes of cancer deaths among women are lung cancer, breast cancer, colon cancer, ovarian cancer and pancreatic cancer. When detected at locally advanced or metastatic stages, no consistently curative treatment regimen exists. Treatment for metastatic cancer includes hormonal ablation, radiation therapy, chemotherapy, hormonal therapy and combination therapies. Unfortunately, there is frequent relapse of an aggressive androgen independent disease that is insensitive to further hormonal manipulation or to treatment with conventional chemotherapy (Ghosh, J. et al. Proc. Natl. Acad. Sci. USA, 95:13182-13187 (1998)). Therefore, there is a need for alternative therapies, such as immunotherapy or reversal of resistance to chemotherapy, radiation therapy, and hormonal therapy. For instance, immunotherapy is predicated on the notion that all drug-resistant tumors should succumb to cytotoxic lymphocyte-mediated killing. Such tumors may also develop cross-resistance to apoptosis-mediated cytotoxic lymphocytes, resulting ultimately in tumor progression and metastasis of the resistant cells (Thompson, C. B. Science, 267:1456-62 (1995)). The mechanism responsible for the anti-apoptotic phenotype, if identified, may be useful as a prognostic and/or diagnostic indicator and target for immunotherapeutic intervention or reversal of resistance to other cytotoxic therapies.

Our recent findings reveal a novel mechanism of tumor cell resistance to immune and non-immune-mediated cytotoxicity. We have shown that resistance to FasL-mediated apoptosis of ovarian and prostate cancer cells is in large part due to the transcription repressor YY1 that inhibits Fas expression. The inhibition of YY1 up-regulates Fas expression and the cells become sensitive to Fas-mediated apoptosis through Fas ligand receptor signaling (Garban, H. J. et al. J. Immunol., 167:75-81 (2001)). In addition, we have also shown that overexpression of YY1 regulates the resistance of tumor cells to tumor necrosis factor-related apoptosis inducing ligand (TRAIL)-induced apoptosis through TRAIL receptor (i.e., DR4 and DR5) signaling (Ng and Bonavida, 2002, Molecular Cancer Therapeutics 1: 1051-1058, Huerta-Yepez, et al., 2004, Oncogene 23:4993-5003). Inhibition of YY1 will also sensitize cancer cells to apoptosis induced by signaling through a TNF-R1 receptor. Also, inhibition of YY1 sensitizes the cancer cells to chemotherapy-induced apoptosis.

YY1 is a multifunctional DNA binding protein, which can activate, repress, or initiate transcription depending on the context in which it binds (Shi, Y. et al. Biochim. Biophys. Acta., 1332:F49-66 (1997)). The transcription factor YY1 has been identified as a potential repressor factor in the human interferon-γ gene (Ye, J. et al. J. Biol. Chem., 269:25728-25734 (1994); Ye, J., et al. Mol. Cell. Biol., 16:4744-4753 (1996)), the IL-3 gene promoter (Ye, J. et al. J. Biol. Chem., 274:26661-26667 (1999)), and the GM-CSF gene promoter (Ye, J. et al. J. Biol. Chem., 269:25728-25734 (1994); Ye, J. et al. Mol. Cell. Biol., 16:157-167 (1996)). Significantly, we have identified a relevant repressor cluster at the silencer region of the human Fas promoter that matched the consensus sequence that binds the transcription factor YY1 (Garban, H. J. et al. J. Immunol., 167:75-81 (2001)). We have also identified a YY1-binding site at the DR5 promoter (Huerta-Yepez, et al., 2005, AACR Abstract).

YY1 is overexpressed in human prostate cancer, lymphoma, myeloma, hepatocarcinoma, and most cancerous tissues as compared to benign tissues. Thus, we consider that YY1 is an important factor in the control of sensitivity of the cells to apoptosis and contributes to tumor progression and metastasis. The mechanism of YY1 overexpression, however, is not known. Very few studies have examined the transcriptional regulation of YY1 (Patten, M. et al. J. Mol. Cell. Cardiol., 32:1341-1352 (2000); Flanagan, J. R. Cell Growth Differ., 6:185-190 (1995)). Our studies show that the signaling pathway for NFkB regulates YY1 expression and DNA binding activity. Accordingly, there is a need for a better understanding of the role of YY1 to tumor progression and therapy-resistant cancers.

### BRIEF SUMMARY OF THE INVENTION

The present invention provides for methods of diagnosing a cancer that overexpresses YY1 in a subject by detecting overexpression of YY1, the method comprising the steps of:
(a) contacting a tissue sample from the subject with an antibody that specifically binds to YY1 protein; and
(b) determining whether or not YY1 protein is overexpressed in the sample; thereby diagnosing the cancer that overexpresses YY1.

The invention further provides for methods of diagnosing a cancer that overexpresses YY1 in a subject by detecting overexpression of YY1, the method comprising the steps of:
(a) contacting a tissue sample from the subject with a primer set of a first oligonucleotide and a second oligonucleotide that each specifically hybridize to YY1 nucleic acid;
(b) amplifying YY1 nucleic acid in the sample; and
(c) determining whether or not YY1 nucleic acid is overexpressed in the sample; thereby diagnosing the cancer that overexpresses YY1.

The invention further provides for methods of providing a prognosis for a cancer that overexpresses YY1 in a subject by detecting overexpression of YY1, the method comprising the steps of:
(a) contacting a tissue sample from the subject with an antibody that specifically binds to YY1 protein; and
(b) determining whether or not YY1 protein is overexpressed in the sample; thereby providing a prognosis for the cancer that overexpresses YY1.

The invention further provides for methods of providing a prognosis for a cancer that overexpresses YY1 in a subject by detecting overexpression of YY1, the method comprising the steps of:
(a) contacting a tissue sample from the subject with a primer set of a first oligonucleotide and a second oligonucleotide that each specifically hybridize to YY1 nucleic acid;
(b) amplifying YY1 nucleic acid in the sample; and
(c) determining whether or not YY1 nucleic acid is overexpressed in the sample; thereby providing a prognosis for the cancer that overexpresses YY1.

Generally, the methods find particular use in diagnosing or providing a prognosis for cancer including prostate cancer, renal cancer, lung cancer, ovarian cancer, breast cancer, colon cancer, leukemias, B-cell lymphomas (e.g., non-Hodgkin's lymphomas, including Burkitt's, Small Cell, and Large Cell lymphomas), hepatocarcinoma or multiple myeloma.

The invention also provides an isolated primer set, the primer set comprising a first oligonucleotide and a second oligonucleotide, each oligonucleotide comprising a nucleotide sequence of 50 nucleotides or less; wherein the first oligonucleotide comprises SEQ ID NO:1 and the second oligonucleotide comprises SEQ ID NO:2.

The invention also provides a method of localizing a cancer that overexpresses YY1 in vivo, the method comprising the step of imaging in a subject a cell overexpressing YY1 polypeptide, thereby localizing the cancer that overexpresses YY1 in vivo.

The invention further provides methods of identifying a compound that inhibits a cancer that overexpresses YY1, the method comprising the steps of:
(a) contacting a cell expressing YY1 polypeptide with a compound; and
(b) determining the effect of the compound on the YY1 polypeptide; thereby identifying a compound that inhibits a cancer that overexpresses YY1. The methods of screening find particular use in identifying compounds that inhibit YY1 expression/activity in cancers such as prostate cancer, renal cancer, ovarian cancer, lung cancer, breast cancer, colon cancer, leukemias, B-cell lymphomas (e.g., non-Hodgkin's lymphomas, including Burkitt's, Small Cell, and Large Cell lymphomas), hepatocarcinoma or multiple myeloma.

The invention further provides methods of treating or inhibiting a cancer that overexpresses YY1 or a therapy resistant cancer in a subject comprising administering to the subject a therapeutically effective amount of one or more YY1 inhibitors. The YY1 inhibitors can be administered alone or concurrently with a conventionally used chemotherapy, radiation therapy, hormonal therapy, or immunotherapy. The methods find particular use in treating prostate cancer, renal cancer, ovarian cancer, lung cancer, breast cancer, colon cancer, leukemias, B-cell lymphomas (e.g., non-Hodgkin's lymphomas, including Burkitt's, Small Cell, and Large Cell lymphomas), hepatocarcinoma, multiple myeloma and other cancers that overexpress YY1 or have YY1-induced immuno and chemo/radio/hormonal resistance to apoptotic-induced stimuli.

### BRIEF DESCRIPTION OF THE DRAWINGS

FIG. 1

Western blot, PC-3 cell line. PC-3 cells were grown in RPMI with 10% of BS. Total cellular protein was extracted from the culture and then separated by SDS-PAGE and transferred onto the nitrocellulose membrane as described in Materials and Methods. The membrane was stained with anti YY1 antibody (1:1500 and 1:3000) or IgG control (1:1500). The β-actin antibody (1:10,000) was used as a loading control. The findings revealed that PC-3 express YY1 constitutively. The blot represents one of two separate experiments

FIG. 2

YY1 Protein Expression in a prostate cancer cell line (PC3). Distinct nuclear and light cytoplasmic staining of YY1 protein is seen by immunohistochemistry (A). Replacing primary anti-YY1 antibody with non-immune pooled rabbit IgG at an equivalent concentration serves as negative control (B), note a complete absence of staining. (Images captured with 40× objective)

FIG. 3

Typical YY1 Protein Expression Localization, Normal Prostate Wholes Tissues. Demonstration of the typical staining pattern of YY1 protein by immunohistochemistry (A), showing predominantly nuclear staining of glandular (thick arrow) and basal cells (thin arrow), as well as stromal fibromuscular cells (triangle). Negative controls include non-immune IgG primary antibody substituted for YY1 (B), and primary YY1 antibody staining after competitive inhibition with immunogen peptide (C). (Images captured with 40× objective)

FIG. 4

Spectrum of YY1 Protein Expression Patterns in Prostate Cancer—Whole Tissues. Immunohistochemical staining for YY1 protein is seen on prostate tissue samples. (A) Normal tissue included for comparison shows crisp diffuse nuclear staining; (C) High grade tumor with finely granular nuclear staining; (E) low grade tumor with nuclear and diffuse cytoplasmic staining, note the normal gland in the lower left (arrow) showing nuclear staining only; (G) High grade tumor with neuroendocrine features showing coarsely granular nuclear and diffuse cytoplasmic staining; (I) low grade tumor with minimal to absent nuclear staining. (B, D, F, H, J are all non-immune pooled rabbit IgG negative controls). (Images captured with 40× objective)

FIG. 5

YY1 Protein Expression Distribution on the Prostate TMA Stratified by Histological Category. Shown are the proportional distributions of YY1 protein staining by immunohistochemistry with attention to the maximal nuclear and cytoplasmic staining intensity, (A and C, respectively), and the total proportion of nuclear and cytoplasmic positivity at any intensity (B and D, respectively) of the target cells of the appropriate histologic category of each spot. 12 informative spots representing metastases are not included here.

FIG. 6

Kaplan Meier Curve for Time to Recurrence. Kaplan Meier Curves for time to tumor recurrence stratified by YY1 protein expression status are depicted for all patients (Gleason case scores 2-9) in (A), and limited to low grade Gleason Scores of 2-6 in (B). Note that in both patient groups a low YY1 expression phenotype is significantly associated with a higher risk to develop recurrent disease. Circles indicate censored patients.

FIG. 7

YY1 Protein Expression in liver tissue arrays. Distinct cytoplasm staining of YY1 protein is seen by immunohistochemistry. The poor cytoplasmic staining is shown in nodular cirrhosisi (A). In contrast, clear cytoplasmic staining is shown in hepatocellular carcinoma (B). (Original magnification: 40×)

FIG. 8

Expression of YY1 in lymphoma tissue arrays. A to C. Appearance of array-tissue spots specimens. A, D, G. Low expression of the lymphoma for YY1. B, E, H. Medium expression. C, F, I. High expression. H. I. High-power view showing specific intracytoplasmic and intranuclear strong expression for YY1.

FIG. 9

Expression of YY1 in lymphoma tissue arrays. A. High-power view showing few malignant cells with specific intracytoplasmic and intranuclear very low immunoexpression for YY1. B. more cells with specific expression. C. spectacular intracytoplasmic and intranuclear immunoexpression in the total malignant cells for YY1 (100×).

FIG. 10

FIG. 10 depicts YY1 expression in different types of bone marrow cells derived from patients with multiple myeloma.

FIG. 11

FIG. 11 depicts the results of a luciferase reporter assay in 293 cells showing luciferase expression after 8 hours stimulation with PMA and serum.

### DETAILED DESCRIPTION OF THE INVENTION

### Introduction

The YY1 transcription factor is a ubiquitously expressed 86-kilodalton zinc finger transcription factor that plays an important role in the regulation of many cellular and viral genes. YY1 functions both as a transcriptional repressor and as a transcriptional activator. YY1 may function as a repressor of genes that regulate tumor cells sensitivity to apoptosis. For instance, we have reported that YY1 inhibits Fas expression and renders cells resistant to Fas ligand-mediated apoptosis and its inhibition results in up-regulation of Fas expression and sensitization to Fas-mediated apoptosis (see, e.g., Garban and Bonavida, JBC, 276(12):8918-8923, 2001; J Immunol., 167:75-81, 2001). We have also found that YY1 regulates the expression of TRAIL receptor DR5 both on the cell surface, as well as intracellularly. YY1 regulates Fas and TRAIL-induced apoptosis in Non-Hodgkin's Lymphoma and other cancers. Further, YY1 regulates the resistance to chemotherapeutic drug-induced (e.g., cisplatin or CDDP) apoptosis in prostate cancer cells and other cancer cells.

Thus, YY1 may inhibit drug/immune-mediated apoptosis, escape of tumor cells from such therapies, selection of cells over-expressing YY1, progression of disease. Alternatively, YY1 may be repressing important tumor suppressor genes, allowing the cells to be transformed and progress to malignancy. In the present application, we examined YY1 in tumor biopsies, by detecting expression in the cytosol and the nuclei by immunohistochemistry, alone, or in association with other markers. Therefore, we have now demonstrated the diagnostic/prognostic significance of YY1 for human prostate cancer and other cancers, including ovarian, renal, lung, lymphomas, myelomas and hepatocarcinomas.

Detection of YY1 is therefore useful for diagnosis and prognosis of prostate cancer as well as other cancers, including ovarian cancer, lung cancer, renal cancer, lymphomas, myelomas and hepatocarcinomas. Detection can include, for example, the level of YY1 mRNA or protein expression, or the localization (i.e., in the nucleus or the cytoplasm) of YY1 mRNA or protein. In terms of early diagnosis, needle, surgical or bone marrow biopsies can be used and examined by immunohistochemistry for expression in cytosol or nuclei, alone or in combination with other markers such as p53, usually negative in prostate cancer and other cancers. Thus, YY1 is a new positive stain that complements the traditional negative stain to enhance the diagnosis of prostate and other cancers. In addition, microlaser microdissection can be used to isolate a few cells and run RT-PCR for YY1 nucleic acid. The following PCT primers can be used to detect YY1: (forward, SEQ ID NO:1) GGC CAC CAC CAC CAC CAC CA; (reverse, SEQ ID NO:2) TTC TTG TTG CCC GGG TCG GC. Molecular imaging can be used to identify individual cells or groups of cells expression specific proteins or enzymatic activity in real time in living patients (Louie et al., 2002). The ability to imaging YY1 could provide a significant role in the localization of cancers within the tissue of a primary tumor and tissues of metastatic tumors. One application of this technique is to help direct the location of needle biopsy sites in the prostate and to assess the extent of cancer within the prostate gland. In addition, the value of imaging systematically provides value for detection of metastatic prostate cancer and other cancers in organs other than the liver and kidney. Finally, cells expressing YY1 can be used for drug discovery to identify new drugs to treat prostate and other cancers, as well as to evaluate prostate and other cancer treatments. Such drugs can be directly used alone or in combination with chemotherapy/immunotherapy to treat prostate cancer and other cancers that are resistant to chemotherapy/immunotherapy. For example, we demonstrate that inhibition of YY1 sensitizes tumor cells to death receptor-induced apoptosis, including TNF-R1, Fas and TRAIL receptors. Therefore YY1 inhibition is useful in sensitizing cancer cells to FasL/TRAIL/TNF-R1 and chemotherapeutic drug-induced apoptosis.

Accordingly, in a first aspect, the invention provides methods of diagnosing a cancer that overexpresses YY1 in a subject by detecting overexpression of YY1, the method comprising the steps of:
(a) contacting a tissue sample from the subject with an antibody that specifically binds to YY1 protein; and
(b) determining whether or not YY1 protein is overexpressed in the sample; thereby diagnosing the cancer that overexpresses YY1. The antibody can be a monoclonal antibody or a polyclonal antibody, but is typically a monoclonal antibody.

In a further aspect, the invention provides methods of diagnosing a cancer that overexpresses YY1 in a subject by detecting overexpression of YY1, the method comprising the steps of:
(a) contacting a tissue sample from the subject with a primer set of a first oligonucleotide and a second oligonucleotide that each specifically hybridize to YY1 nucleic acid;
(b) amplifying YY1 nucleic acid in the sample; and
(c) determining whether or not YY1 nucleic acid is overexpressed in the sample; thereby diagnosing the cancer that overexpresses YY1. In one embodiment, the first oligonucleotide comprises SEQ ID NO:1 and the second oligonucleotide comprises SEQ ID NO:2.

In a further aspect, the invention provides methods of providing a prognosis for a cancer that overexpresses YY1 in a subject by detecting overexpression of YY1, the method comprising the steps of:
(a) contacting a tissue sample from the subject with an antibody that specifically binds to YY1 protein; and
(b) determining whether or not YY1 protein is overexpressed in the sample; thereby providing a prognosis for the cancer that overexpresses YY1. The antibody can be a monoclonal antibody or a polyclonal antibody, but is typically a monoclonal antibody.

In a further aspect, the invention provides methods of providing a prognosis for a cancer that overexpresses YY1 in a subject by detecting overexpression of YY1, the method comprising the steps of:
(a) contacting a tissue sample from the subject with a primer set of a first oligonucleotide and a second oligonucleotide that each specifically hybridize to YY1 nucleic acid;
(b) amplifying YY1 nucleic acid in the sample; and
(c) determining whether or not YY1 nucleic acid is overexpressed in the sample; thereby providing a prognosis for the cancer that overexpresses YY1. In one embodiment, the first oligonucleotide comprises SEQ ID NO:1 and the second oligonucleotide comprises SEQ ID NO:2.

The diagnosis and prognosis methods can also be carried out by determining the extent of YY1 protein from a cancer patient binds to a DNA sequence comprising a YY1 binding sequence. The diagnosis and prognosis methods can also be carried out by determining whether or not a YY1 protein is localized in the nucleus or the cytoplasm of a cell, wherein YY1 localization in the nucleus indicates a cancerous phenotype. The diagnosis and prognosis methods can also be carried out by determining whether or not the YY1 protein is full-length or truncated.

In determining the levels of protein expression or the localization of YY1 protein, polyclonal or monoclonal antibodies that specifically bind YY1 can be used.

Generally, the methods find particular use in diagnosing or providing a prognosis for prostate cancer, ovarian cancer, lung cancer, renal cancer, breast cancer, colon cancer, leukemias, B-cell lymphomas (e.g., non-Hodgkin's lymphomas, including Burkitt's, Small Cell, and Large Cell lymphomas), hepatocarcinoma or multiple myeloma. In carrying out the diagnosis or prognosis methods, the determination of whether or not the YY1 is overexpressed, optionally can be made by comparing the test biological sample to a control autologous biological sample from normal tissue.

In some embodiments, the methods of diagnosis or prognosis are carried out by determining the extent by which the YY1 protein binds to DNA compared to YY1 from normal tissue, for example, by employing an electrophoretic mobility shift assay (EMSA).

In carrying out the diagnosis or prognosis methods, the tissue sample can be taken from a tissue of the primary tumor or a metastatic tumor. A tissue sample can be taken, for example, by an excisional biopsy, an incisional biopsy, a needle biopsy, a surgical biopsy, a bone marrow biopsy or any other biopsy technique known in the art. In some embodiments, the tissue sample is microlaser microdissected cells from a needle biopsy. In some embodiments, the tissue sample is a metastatic cancer tissue sample. In some embodiments, the tissue sample is fixed, for example, with paraformaldehyde, and embedded, for example, in paraffin. For example, the tissue sample can be from cancers such as prostate, ovary, lung, colon, breast, etc. and from the blood, serum, saliva, urine, bone, lymph node, liver or kidney tissue.

In another aspect, the invention also provides an isolated primer set, the primer set comprising a first oligonucleotide and a second oligonucleotide, each oligonucleotide comprising a nucleotide sequence of 50 nucleotides or less; wherein the first oligonucleotide comprises SEQ ID NO:1 and the second oligonucleotide comprises SEQ ID NO:2.

In another aspect, the invention also provides methods of localizing a cancer that overexpresses YY1 in vivo, the method comprising the step of imaging in a subject a cell overexpressing YY1 polypeptide, thereby localizing the cancer that overexpresses YY1 in vivo. The methods find particular use in diagnosing or providing a prognosis for cancers such as prostate cancer, ovarian cancer, lung cancer, renal cancer, breast cancer, colon cancer, leukemias, B-cell lymphomas (e.g., non-Hodgkin's lymphomas, including Burkitt's, Small Cell, and Large Cell lymphomas), hepatocarcinoma or multiple myeloma.

In another aspect, the invention further provides methods of identifying a compound that inhibits a cancer that overexpresses YY1, the method comprising the steps of:
(a) contacting a cell expressing YY1 polypeptide with a compound; and
(b) determining the effect of the compound on the YY1 polypeptide; thereby identifying a compound that inhibits a cancer that overexpresses YY1.

In another aspect, the invention further provides methods of identifying a compound that inhibits a therapy resistant cancer, the method comprising the steps of:
(a) contacting a cell expressing YY1 polypeptide with a compound; and
(b) determining the effect of the compound on the YY1 polypeptide; thereby identifying a compound that inhibits a therapy resistant cancer, when used alone or in combination with cytotoxic therapies.

In carrying out the methods of screening, the compound can be, for example, a small organic molecule, a polypeptide, an antibody, a polynucleotide, an inhibitory RNA, including an siRNA. In some embodiments, the compound inhibits YY1 expression, for example, transcription or translation. In some embodiments, the compound inhibits YY1 transcription by inhibiting transcription factors such as NF-κB. In some embodiments, the compound inhibits YY1 function, for example, in transcriptional activity. In some embodiments, the compound inhibits the binding of YY1 to a DNA sequence. In some embodiments, the compound inhibits YY1 binding to other proteins, including other transcription factors. In some embodiments, the compound sensitizes the cell to apoptosis induced by cell signaling through a death receptor (i.e., Fas ligand receptor, TRAIL receptor, TNF-R1) or through conventional cytotoxic therapies. In some embodiments, the compound inhibits YY1 mRNA. In some embodiments, the compound accelerates the degradation of YY1 via the proteasome system.

Typically, the compound will inhibit a cancer that overexpresses YY1 or a therapy resistant cancer in combination with another cancer treatment, for example, co-administration with a death receptor agonist or another chemotherapeutic agent known in the art. Compounds of interest that inhibit YY1 sensitize cancer cells to conventional cancer treatments, including chemotherapy, radiotherapy, hormonal therapy, immunotherapy and other methods of treating cancer.

Generally, the methods of screening find particular use in identifying compounds that inhibit cancers such as prostate cancer, ovarian cancer, lung cancer, renal cancer, breast cancer, colon cancer, leukemias, B-cell lymphomas (e.g., non-Hodgkin's lymphomas, including Burkitt's, Small Cell, and Large Cell lymphomas), hepatocarcinoma or multiple myeloma. In one embodiment the cell comprises a promoter sequence bound by YY1 operably linked to a reporter nucleic acid, for example, firefly luciferase or green fluorescent protein.

In another aspect, the invention further provides methods of treating or inhibiting a cancer that overexpresses YY1 or a therapy resistant cancer in a subject comprising administering to the subject a therapeutically effective amount of one or more YY1 inhibitors.

In another aspect, the invention provides for methods of sensitizing a tumor to conventional cancer treatments, including chemotherapy, radiation therapy, hormonal therapy, and immunotherapy, comprising administering to the subject a therapeutically effective amount of one or more YY1 inhibitors.

The YY1 inhibitor can be a known compound, for example, an NO donor, an antimitotic drug, an inhibitory RNA sequence (i.e., siRNA or antisense RNA), an antibody such as CD20 (including antibody molecules that specifically bind CD20, e.g., rituximab), or combinations thereof. The YY1 inhibitor can be identified according to the screening methods of the present invention.

In carrying out the methods of treatment, the one or more YY1 inhibitors can be administered concurrently with conventional therapies, for example, currently used chemotherapy, radiation therapy, hormonal therapy or immunotherapy treatments. In one embodiment, the YY1 inhibitor is co-administered with a second pharmacological agent, for example, an agonist of a death receptor, including a Fas ligand receptor, a TRAIL receptor or TNF-R1. In one embodiment, the YY1 inhibitor is co-administered with an agonist of a death receptor, for example, a Fas ligand receptor (e.g., Fas), a TRAIL receptor (e.g., DR4 or DR5) or TNF-R1. The agonist can be an antibody, including a monoclonal antibody or a polyclonal antibody. In one embodiment, the YY1 inhibitor is co-administered with a monoclonal antibody against a DR5 receptor. In one embodiment, the YY1 inhibitor is coadministered with a TRAIL polypeptide.

The one or more YY1 inhibitors can be co-administered simultaneously or sequentially with another therapeutic agent. In one embodiment, one or more YY1 inhibitors are administered prior to administering another therapeutic agent. This strategy can establish a sensitizing effect on the cell before administering a cytotoxic agent. In one embodiment, the YY1 is co-administered with a conventional chemotherapeutic agent, for example, cisplatin (CDDP), VP16, adriamycin (doxorubicin hydrochloride), vincristine, 5-fluorouracil, etc.

In one embodiment, the YY1 inhibitor is an NO donor. In one embodiment, the NO donor is selected from the group consisting of L-arginine, amyl nitrite, isoamyl nitrite, nitroglycerin, isosorbide dinitrate, isosorbide-2-mononitrate, isosorbide-5-mononitrate, erythrityl tetranitrate, pentaerythritol tetranitrate, sodium nitroprusside, 3 morpholinosydnonimine, molsidomine, N-hydroxyl-L-arginine, S,S-dinitrosodthiol, ethylene glycol dinitrate, isopropyl nitrate, glyceryl-1-mononitrate, glyceryl-1,2-dinitrate, glyceryl-1,3-dinitrate, glyceryl trinitrate, butane-1,2,4-triol trinitrate, N,O diacetyl-N-hydroxy-4-chlorobenzenesulfonamide, NG hydroxy-L-arginine, hydroxyguanidine sulfate, (±)-S-nitroso-N-acetylpenicillamine, S nitrosoglutathione, (±)-(E)-ethyl-2-[(E)-hydroxyimino]-5-nitro-3-hexeneamide (FK409), (±)-N-[(E)-4-ethyl-3-[(Z)-hydroxyimino]-5-nitro-3-hexen-1-yl]-3-pyridinecarboxamide (FR144420), 4-hydroxymethyl-3-furoxancarboxamide, (Z)-1-[2-(2-Aminoethyl)-N-(2-ammonioethyl)amino]diazen-1-ium-1,2-diolate; NOC-18; 3,3-bis(aminoethyl)-1-hydroxy-2-oxo-*1-triazene (DETA/NONOate), NO gas, and mixtures thereof. In one embodiment, the NO donor is a conjugate with another compound, for example, aspirin, a cytotoxic drug or an antibody. Nitric oxide reducing drugs as described, for example, in Nappoli, et al., Annual Review Pharmacology and Toxicology (2003) 43:97-123; and Ignarro, et al., Circulation Research (2002) 90:21-28.

In one embodiment, the YY1 inhibitor is an activator of inducible nitric oxide synthase (iNOS). Exemplified activators of iNOS include cytokines, for example, IL-1β, and interferons (IFN), including IFN-α, IFN-β, and IFN-γ.

In one embodiment the YY1 inhibitor is an inhibitory RNA, for example, an small inhibitory RNA (siRNA) or an antisense RNA (asRNA). siRNA molecules for inhibiting a target gene of interest can be purchased commercially, for example, from Santa Cruz Biotechnology, Santa Cruz, Calif. and SuperArray Bioscience Corp., Frederick, Md.

In one embodiment, the YY1 inhibitor inhibits YY1 transcription. For example, inhibitors of NF-κB and the NF-κB pathway inhibit YY1 transcription. Chemotherapeutic drugs, including cisplatin (CDDP) and adriamycin (doxorubicin hydrochloride), also inhibit YY1 transcription via inhibition of NF-κB.

In one embodiment, the YY1 inhibitor is an antimitotic drug. In one embodiment, the antimitotic drug is selected from the group consisting of vinca alkaloids and taxanes, or combinations thereof. In one embodiment, the vinca alkaloid is selected from the group consisting of vinblastine, vincristine, vindesine, vinorelbine, and combinations thereof. In one embodiment, the taxane is selected from the group consisting of paclitaxel, docetaxel, and combinations thereof. In one embodiment the antimitotic drug is 2-methoxyestradiol.

### DEFINITIONS

“YY1” refers to nucleic acids, e.g., gene, pre-mRNA, mRNA, and polypeptides, polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to a polypeptide encoded by a referenced nucleic acid or an amino acid sequence described herein; (2) specifically bind to antibodies, e.g., polyclonal antibodies, raised against an immunogen comprising a referenced amino acid sequence, immunogenic fragments thereof, and conservatively modified variants thereof; (3) specifically hybridize under stringent hybridization conditions to a nucleic acid encoding a referenced amino acid sequence, and conservatively modified variants thereof; (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a reference nucleic acid sequence. A polynucleotide or polypeptide sequence is typically from a mammal including, but not limited to, primate, e.g., human; rodent, e.g., rat, mouse, hamster; cow, pig, horse, sheep, or any mammal. The nucleic acids and proteins of the invention include both naturally occurring or recombinant molecules. The gene for YY1 is provided, for example, by Accession Nos. NM_{—}003403, BC037308, M76541, BC065366, and Z14077; the protein sequence is provided, for example, by Accession Nos. NP_{—}003394, AAA59467, AAA59926, and XP_{—}510162. Truncated and alternatively spliced forms of YY1 are included in the definition of YY1. Truncated forms of YY1 have been described by Krippner-Heidenreich, Mol Cell Biol (2005) 25:3704; Begon, et al., J Biol Chem (2005) 280:24428; Nishiyama, et al., Biosciences, Biotech, Biochem (2003) 67:654; and Berndt, et al., J Neurochem (2001) 77:935.

“Cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, esophagus, and liver cancer, including hepatocarcinoma, lymphoma, including non-Hodgkin's lymphomas (e.g., Burkitt's, Small Cell, and Large Cell lymphomas) and Hodgkin's lymphoma, leukemia, and multiple myeloma.

“Therapy resistant” cancers, tumor cells, and tumors refers to cancers that have become resistant to both apoptosis-mediated (e.g., through death receptor cell signaling, for example, Fas ligand receptor, TRAIL receptors, TNF-R1, chemotherapeutic drugs, radiation) and non-apoptosis mediated (e.g., toxic drugs, chemicals) cancer therapies, including chemotherapy, hormonal therapy, radiotherapy, and immunotherapy.

“Therapeutic treatment” and “cancer therapies” refers to apoptosis-mediated and non-apoptosis mediated cancer therapies, including chemotherapy, hormonal therapy, radiotherapy, and immunotherapy. Cancer therapies can be enhanced by concurrent administration with a sensitizing agent, for example, an inhibitor of YY1.

The terms “overexpress,” “overexpression” or “overexpressed” interchangeably refer to a gene that is transcribed or translated at a detectably greater level, usually in a cancer cell, in comparison to a normal cell. Overexpression therefore refers to both overexpression of XIAP protein and RNA (due to increased transcription, post transcriptional processing, translation, post translational processing, altered stability, and altered protein degradation), as well as local overexpression due to altered protein traffic patterns (increased nuclear localization), and augmented functional activity, e.g., as a transcription factor, as a DNA binding factor. Overexpression can be detected using conventional techniques for detecting mRNA (i.e., RT-PCR, PCR, hybridization) or proteins (i.e., ELISA, Western blots, flow cytometry, immunofluorescence, immunohistochemical, DNA binding assay techniques). Overexpression can be 10%, 20%, 30%, 40%, 50%, 60%, 70%, 80%, 90% or more in comparison to a normal cell. In certain instances, overexpression is 1-fold, 2-fold, 3-fold, 4-fold or more higher levels of transcription or translation in comparison to a normal cell.

The terms “cancer that overexpresses YY1” and “cancer associated with the overexpression of YY1” interchangeably refer to cancer cells or tissues that overexpress YY1, in accordance with the above definition. These terms can also refer to YY1-mediated resistance to apoptosis through death receptors (TNF-R1, Fas ligand receptors, TRAIL receptors), optionally in combination with the administration of chemotherapeutic drugs, radiation therapy or hormonal therapy.

The terms “cancer-associated antigen” or “tumor-specific marker” or “tumor marker” interchangeably refers to a molecule (typically protein, carbohydrate or lipid) that is preferentially expressed in a cancer cell in comparison to a normal cell, and which is useful for the preferential targeting of a pharmacological agent to the cancer cell. A marker or antigen can be expressed on the cell surface or intracellularly. Oftentimes, a cancer-associated antigen is a molecule that is overexpressed or stabilized with minimal degradation in a cancer cell in comparison to a normal cell, for instance, 1-fold over expression, 2-fold overexpression, 3-fold overexpression or more in comparison to a normal cell. Oftentimes, a cancer-associated antigen is a molecule that is inappropriately synthesized in the cancer cell, for instance, a molecule that contains deletions, additions or mutations in comparison to the molecule expressed on a normal cell. Oftentimes, a cancer-associated antigen will be expressed exclusively in a cancer cell and not synthesized or expressed in a normal cell. Exemplified cell surface tumor markers include the proteins c-erbB-2 and human epidermal growth factor receptor (HER) for breast cancer, PSMA for prostate cancer, and carbohydrate mucins in numerous cancers, including breast, ovarian and colorectal. Exemplified intracellular tumor markers include, for example, mutated tumor suppressor or cell cycle proteins, including p53.

An “agonist” refers to an agent that binds to a polypeptide or polynucleotide of the invention, stimulates, increases, activates, facilitates, enhances activation, sensitizes or up regulates the activity or expression of a polypeptide or polynucleotide of the invention.

An “antagonist” refers to an agent that inhibits expression of a polypeptide or polynucleotide of the invention or binds to, partially or totally blocks stimulation, decreases, prevents, delays activation, inactivates, desensitizes, or down regulates the activity of a polypeptide or polynucleotide of the invention.

“Inhibitors,” “activators,” and “modulators” of expression or of activity are used to refer to inhibitory, activating, or modulating molecules, respectively, identified using in vitro and in vivo assays for expression or activity, e.g., ligands, agonists, antagonists, and their homologs and mimetics. The term “modulator” includes inhibitors and activators. Inhibitors are agents that, e.g., inhibit expression, e.g., translation, post-translational processing, stability, degradation, or nuclear or cytoplasmic locallization of a polypeptide, or transcription, post transcriptional processing, stability or degradation of a polynucleotide of the invention or bind to, partially or totally block stimulation, DNA binding, transcription factor activity or enzymatic activity, decrease, prevent, delay activation, inactivate, desensitize, or down regulate the activity of a polypeptide or polynucleotide of the invention, e.g., antagonists. Activators are agents that, e.g., induce or activate the expression of a polypeptide or polynucleotide of the invention or bind to, stimulate, increase, open, activate, facilitate, enhance activation, DNA binding or enzymatic activity, sensitize or up regulate the activity of a polypeptide or polynucleotide of the invention, e.g., agonists. Modulators include naturally occurring and synthetic ligands, antagonists, agonists, small chemical molecules, antibodies, inhibitory RNA molecules (i.e., siRNA or antisense RNA) and the like. Assays to identify inhibitors and activators include, e.g., applying putative modulator compounds to cells, in the presence or absence of a polypeptide or polynucleotide of the invention and then determining the functional effects on a polypeptide or polynucleotide of the invention activity. Samples or assays comprising a polypeptide or polynucleotide of the invention that are treated with a potential activator, inhibitor, or modulator are compared to control samples without the inhibitor, activator, or modulator to examine the extent of effect. Control samples (untreated with modulators) are assigned a relative activity value of 100%. Inhibition is achieved when the activity value of a polypeptide or polynucleotide of the invention relative to the control is about 80%, optionally 50% or 25-1%. Activation is achieved when the activity value of a polypeptide or polynucleotide of the invention relative to the control is 110%, optionally 150%, optionally 200-500%, or 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, RNAi, oligonucleotide, etc. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 Daltons and less than about 2500 Daltons, preferably less than about 2000 Daltons, preferably between about 100 to about 1000 Daltons, more preferably between about 200 to about 500 Daltons.

The term “nitric oxide donor” or “NO donor” refers to any compound capable of the intracellular delivery of nitric oxide. Typically, an NO donor is any compound capable of denitrition that releases nitric oxide. Also included are those compounds that can be metabolized in vivo into a compound which delivers nitric oxide (e.g., a prodrug form of a NO donor). An NO donor can be a synthetic or naturally occurring organic chemical compound and can be a polypeptide. Exemplified pharmaceutical agents that are NO donors include arginine (L- and D-), amyl nitrite, isoamyl nitrite, nitroglycerin, isosorbide dinitrate, isosorbide-5-mononitrate, erythrityl tetranitrate. Nitric oxide synthases, both constitutive and inducible forms, are also nitric oxide donors.

The term “inducer of inducible nitric oxide synthase (iNOS)” or “activator of iNOS” refers to any compound that promotes the expression (transcription or translation) and/or promotes that catalytic activity of iNOS.

A “cell-cycle-specific” or “antimitotic” or “cytoskeletal-interacting” drug interchangeably refer to any pharmacological agent that blocks cells in mitosis. Generally, cell-cycle-specific-drugs bind to the cytoskeletal protein tubulin and block the ability of tubulin to polymerize into microtubules, resulting in the arrest of cell division at metaphase. Exemplified cell-cycle-specific drugs include vinca alkaloids, taxanes, colchicine, and podophyllotoxin. Exemplified vinca alkaloids include vinblastine, vincristine, vindesine and vinorelbine. Exemplifed taxanes include paclitaxel and docetaxel. Another example of a cytoskeletal-interacting drug includes 2-methoxyestradiol.

An “siRNA” or “RNAi” refers to a nucleic acid that forms a double stranded RNA, which double stranded RNA has the ability to reduce or inhibit expression of a gene or target gene when the siRNA expressed in the same cell as the gene or target gene. “siRNA” or “RNAi” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 or about 24-29 nucleotides in length, e.g., 20, 21, 22, 23, 24, 25, 26, 27, 28, 29, or 30 nucleotides in length.

“Determining the functional effect” refers to assaying for a compound that increases or decreases a parameter that is indirectly or directly under the influence of a polynucleotide or polypeptide of the invention, e.g., measuring physical and chemical or phenotypic effects. Such functional effects can be measured by any means known to those skilled in the art, e.g., changes in spectroscopic (e.g., fluorescence, absorbance, refractive index), hydrodynamic (e.g., shape), chromatographic, or solubility properties for the protein; measuring inducible markers or transcriptional activation of the protein; measuring binding activity or binding assays, e.g. binding to antibodies, binding to DNA; measuring changes in ligand binding affinity; measurement of calcium influx; measurement of the accumulation of an enzymatic product of a polypeptide of the invention or depletion of an substrate; changes in enzymatic activity, e.g., kinase activity, measurement of changes in protein levels of a polypeptide of the invention; measurement of RNA stability; G-protein binding; GPCR phosphorylation or dephosphorylation; signal transduction, e.g., receptor-ligand interactions, second messenger concentrations (e.g., cAMP, IP3, or intracellular Ca2+); identification of downstream or reporter gene expression (CAT, luciferase, β-gal, GFP and the like), e.g., via chemiluminescence, fluorescence, colorimetric reactions, antibody binding, inducible markers, and ligand binding assays.

Samples or assays comprising a nucleic acid or protein disclosed herein that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition is achieved when the activity value relative to the control is about 80%, preferably 50%, more preferably 25-0%. Activation is achieved when the activity value relative to the control (untreated with activators) is 110%, more preferably 150%, more preferably 200-500% (i.e., two to five fold higher relative to the control), more preferably 1000-3000% higher.

“Biological sample” includes sections of tissues such as biopsy and autopsy samples, and frozen sections taken for histologic purposes. Such samples include blood and blood fractions or products (e.g., serum, plasma, platelets, red blood cells, and the like), sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, Mouse; rabbit; or a bird; reptile; or fish.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (i.e., prostate, lymph node, liver, bone marrow, blood cell), the size and type of the tumor (i.e., solid or suspended (i.e., blood or ascites)), among other factors. Representative biopsy techniques include excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy” of the tumor mass, or a “fine-needle aspiration biopsy” which generally obtains a suspension of cells from within the tumor mass. Biopsy techniques are discussed, for example, in Harrison's Principles of Internal Medicine, Kasper, et al., eds., 16th ed., 2005, Chapter 70, and throughout Part V.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat.'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1987-2005, Wiley Interscience)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants” and nucleic acid sequences encoding truncated forms of YY1. Similarly, a particular protein encoded by a nucleic acid implicitly encompasses any protein encoded by a splice variant or truncated form of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. Nucleic acids can be truncated at the 5′ end or at the 3′ end. Polypeptides can be truncated at the N-terminal end or the C-terminal end. Truncated versions of nucleic acid or polypeptide sequences can be naturally occurring or recombinantly created. Truncated forms of YY1 are described, for example, in Begon, et al, J Biol Chem (2005) 280:24428; Krippner-Heidenreich, et al., Mol Cell Biol (2005) 25:3704; Nishiyama, et al., Biosci Biotechnol Biochem (2003) 67:654; and Berndt, et al., J Neurochem (2001) 77:935.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., supra.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

In one embodiment, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

By “therapeutically effective amount or dose” or “sufficient amount or dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

### Assays for Modulators of YY1

Modulation of a YY1, and corresponding modulation of cellular, e.g., tumor cell, proliferation, can be assessed using a variety of in vitro and in vivo assays, including cell-based models. Such assays can be used to test for inhibitors and activators of YY1 transcription or translation, or YY1 protein activity, and consequently, inhibitors and activators of cellular proliferation, including modulators of chemotherapeutic and immunotherapeutic sensitivity and toxicity. Assays for modulation of YY1 include cell-viability, cell proliferation, cell responses to apoptotic stimuli, gene transcription, mRNA arrays, kinase or phosphatase activity, interaction with other proteins including other transcription factors, and DNA binding. Such modulators of YY1 are useful for treating disorders related to pathological cell proliferation, e.g., cancer, autoimmunity, aging. Modulators of YY1 activity can be tested using in vivo well cells expressing YY1 and in vitro well, either recombinant or naturally occurring YY1 protein, preferably human YY1. Wild type YY1 as well as truncated and alternatively spliced forms of YY1 are useful targets.

Measurement of cellular proliferation by modulation with a YY1 protein or a YY1 nucleic acid, either recombinant or naturally occurring, can be performed using a variety of assays, in vitro, in vivo, and ex vivo, as described herein. A suitable physical, chemical or phenotypic change that affects activity, e.g., enzymatic activity such as kinase activity, cell proliferation, or ligand binding (e.g., a YY1 protein or nucleic acid receptor) can be used to assess the influence of a test compound on the polypeptide of this invention. When the functional effects are determined using intact cells or animals, one can also measure a variety of effects, such as, ligand binding, DNA binding, kinase activity, transcriptional changes to both known and uncharacterized genetic markers (e.g., northern blots), changes in cell metabolism, changes related to cellular proliferation, cell surface marker expression, DNA synthesis, marker and dye dilution assays (e.g., GFP and cell tracker assays), contact inhibition, tumor growth in nude mice, etc.

In Vitro Assays

Assays to identify compounds with YY1 modulating activity can be performed in vitro. Such assays can use a full length YY1 protein or a variant thereof, or a mutant thereof, a truncated form or a fragment of a YY1 protein. Purified recombinant or naturally occurring YY1 protein can be used in the in vitro methods of the invention. In addition to purified YY1 protein, the recombinant or naturally occurring YY1 protein can be part of a cellular lysate or a cell membrane. As described below, the binding assay can be either solid state or soluble. Preferably, the protein or membrane is bound to a solid support, either covalently or non-covalently. Often, the in vitro assays of the invention are substrate or ligand binding or affinity assays, either non-competitive or competitive. Other in vitro assays include measuring changes in spectroscopic (e.g., fluorescence, absorbance, refractive index), hydrodynamic (e.g., shape), chromatographic, or solubility properties for the protein. Other in vitro assays include enzymatic activity assays, such as phosphorylation or autophosphorylation assays). Preferred in vitro assay systems include DNA binding assays (EMSA), using cells that overexpress YY1, and using cells having a promoter comprising a YY1 binding sequence operably linked to a reporter gene.

In one embodiment, a high throughput binding assay is performed in which the YY1 protein, a truncated form or a fragment thereof is contacted with a potential modulator and incubated for a suitable amount of time. In one embodiment, the potential modulator is bound to a solid support, and the YY1 protein is added. In another embodiment, the YY1 protein is bound to a solid support. A wide variety of modulators can be used, as described herein, including small organic molecules, peptides, antibodies, and YY1 binding protein or nucleic acid analogs. A wide variety of assays can be used to identify YY1-modulator binding, including labeled protein-protein binding assays, electrophoretic mobility shifts, immunoassays, enzymatic assays such as kinase assays, and the like. In some cases, the binding of the candidate modulator is determined through the use of competitive binding assays, where interference with binding of a known ligand or substrate is measured in the presence of a potential modulator.

In one embodiment, microtiter plates are first coated with either a YY1 protein or a YY1 binding protein (i.e. anti-YY1 antibody, transcription factors) or nucleic acid, and then exposed to one or more test compounds potentially capable of inhibiting the binding of a YY1 protein to a YY1 binding protein or nucleic acid. A labeled (i.e., fluorescent, enzymatic, radioactive isotope) binding partner of the coated protein, either a YY1 binding protein or nucleic acid, or a YY1 protein, is then exposed to the coated protein and test compounds. Unbound protein (or nucleic acid) is washed away as necessary in between exposures to a YY1 protein, a YY1 binding protein or nucleic acid, or a test compound. An absence of detectable signal indicates that the test compound inhibited the binding interaction between a YY1 protein and a YY1 binding protein or nucleic acid. The presence of detectable signal (i.e., fluorescence, colorimetric, radioactivity) indicates that the test compound did not inhibit the binding interaction between a YY1 protein and a YY1 binding protein or nucleic acid. One can also use chromatographic techniques, for example HPLC, and evaluate elution profiles of YY1 alone and YY1 complexed with other factors, including DNA and/or other transcription factors. The presence or absence of detectable signal is compared to a control sample that was not exposed to a test compound, which exhibits uninhibited signal. In some embodiments the binding partner is unlabeled, but exposed to a labeled antibody that specifically binds the binding partner.

Cell-Based In Vivo Assays

In another embodiment, YY1 protein is expressed in a cell, and functional, e.g., physical and chemical or phenotypic, changes are assayed to identify YY1 and modulators of cellular proliferation, e.g., tumor cell proliferation. Cells expressing YY1 proteins can also be used in binding assays and enzymatic assays. Preferably, the cells overexpress or under express YY1 in comparison to a normal cell of the same type. Any suitable functional effect can be measured, as described herein. For example, cellular morphology (e.g., cell volume, nuclear volume, cell perimeter, and nuclear perimeter), ligand binding, kinase activity, apoptosis, cell surface marker expression, cellular proliferation, cellular localization of YY1 proteins or transcripts, DNA binding by YY1, GFP positivity and dye dilution assays (e.g., cell tracker assays with dyes that bind to cell membranes), DNA synthesis assays (e.g., ³H-thymidine and fluorescent DNA-binding dyes such as BrdU or Hoechst dye with FACS analysis), are all suitable assays to identify potential modulators using a cell based system. Suitable cells for such cell based assays include both primary cancer or tumor cells and cell lines, as described herein, e.g., A549 (lung), MCF7 (breast, p53 wild-type), H1299 (lung, p53 null), Hela (cervical), PC3 (prostate, p53 mutant), MDA-MB-231 (breast, p53 wild-type). Variants derived from these cell lines with specific gene modification will also be used. Cancer cell lines can be p53 mutant, p53 null, or express wild type p53. The YY1 protein can be naturally occurring or recombinant. Also, truncated forms or fragments of YY1 or chimeric YY1 proteins can be used in cell based assays.

Cellular YY1 polypeptide levels can be determined by measuring the level of protein or mRNA. The level of YY1 protein or proteins related to YY1 are measured using immunoassays such as western blotting, ELISA, immunofluorescence and the like with an antibody that selectively binds to the YY1 polypeptide or a fragment thereof. For measurement of mRNA, amplification, e.g., using PCR, RT-PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein. It is also useful to observe YY1 protein translocation into the nucleus and other cellular compartments by, for example, confocal microscopy. YY1 binding to DNA can be evaluated with electrophoretic mobility shift assays (EMSA). Typically, the YY1 protein is purified for use in EMSA, but need not be. YY1 interaction with other proteins, including other transcription factors, can be measured using standard immunoprecipitation and immunoblotting techniques. YY1 binding to other factors, either DNA or protein, can be evaluated by labeling YY1 protein, for example, with a fluorochrome.

Alternatively, YY1 expression can be measured using a reporter gene system. Such a system can be devised using an YY1 protein promoter which modulates transcription of YY1, or a YY1 responsive site, which is modulated by binding of YY1, operably linked to a reporter gene, including chloramphenicol acetyltransferase, firefly luciferase, bacterial luciferase, β-galactosidase, green fluorescent protein (GFP) and alkaline phosphatase. Furthermore, the protein of interest can be used as an indirect reporter via attachment to a second reporter such as red or green fluorescent protein (see, e.g., Mistili & Spector, Nature Biotechnology 15:961-964 (1997)). Exemplified YY1 responsive sites can be located, for example, in the promoters for ornithine decarboxylase antizyme, death receptor 5 (DR5), and Fas. The reporter construct is typically transfected into a cell. After treatment with a potential modulator, the amount of reporter gene transcription, translation, or activity is measured according to standard techniques known to those of skill in the art. In a preferred embodiment, plasmids that allow for stable transfection are used.

One reporter gene system of use in the present invention utilizes GFP fluorescence as the reporter signal. In this reporter system, a human Ornithine Decarboxylase Antizyme 1 (ODA1) minimal promoter (Hayashi T., et al. (1997) Gene 203:131-9) containing 201 bp upstream of the translation initiation site that includes an unique wild type responsive site (cgccattttgcga) for YY1 is ligated to a GFP reporter vector (e.g., GFP-based pGlow-TOPO®, Invitrogen, Carlsbad, Calif.). A wild-type YY1 responsive site can be used in parallel with a mutated YY1 responsive site, for example, where a YY1 cis-acting element (cgttgttttgcga) is mutated. GFP-based reporter activity from transfected cells with these constructs can be analyzed by direct fluorescence emission at 510 nm using excitation at 395 nm in a Fluorometer (Perkin Elmer Applied Biosystems, Foster City, Calif.).

Another reporter gene system of use in the present invention utilized firefly luciferase luminescence as the reporter signal. In this reporter system, a wild-type DR5 promoter is operably linked to a luciferase reporter sequence. The wild-type DR5YY1 responsive site can be used in parallel with a DR5 promoter having a non-functional YY1 responsive site, for example, a 5′-deletion mutant-605 that includes the YY1 binding site (pDR5/-605) (described in Yoshida, et al, (2001) FEBS Letters, 507:381-385), or a DR5 promoter sequence missing the YY1 binding sequence (pDR5-YY1 mutant) (see, Huerta-Yepez, et al., AACR Abstract, 2005).

Animal Models

Animal models of cellular proliferation also find use in screening for modulators of cellular proliferation. Similarly, transgenic animal technology including gene knockout technology, for example, as a result of homologous recombination with an appropriate gene targeting vector, or gene overexpression, will result in the absence or increased expression of the YY1 protein. The same technology can also be applied to make knock-out cells. When desired, tissue-specific expression or knockout of the YY1 protein may be necessary. Transgenic animals generated by such methods find use as animal models of cellular proliferation and are additionally useful in screening for modulators of cellular proliferation.

Knock-out cells and transgenic mice can be made by insertion of a marker gene or other heterologous gene into an endogenous YY1 gene site in the mouse genome via homologous recombination. Such mice can also be made by substituting an endogenous YY1 with a mutated version of the YY1 gene, or by mutating an endogenous YY1, e.g., by exposure to carcinogens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988), Teratocarcinomas and Embryonic Stem Cells: A Practical Approach, Robertson, ed., IRL Press, Washington, D.C., (1987), and Pinkert, Transgenic Animal Technology: A Laboratory Handbook, Academic Press (2003).

Exemplary Assays

Soft Agar Growth or Colony Formation in Suspension

Normal cells require a solid substrate to attach and grow. When the cells are transformed, they lose this phenotype and grow detached from the substrate. For example, transformed cells can grow in stirred suspension culture or suspended in semi-solid media, such as semi-solid or soft agar. The transformed cells, when transfected with tumor suppressor genes, regenerate normal phenotype and require a solid substrate to attach and grow.

Soft agar growth or colony formation in suspension assays can be used to identify YY1 modulators. Typically, transformed host cells (e.g., cells that grow on soft agar) are used in this assay. For example, RKO or HCT116 cell lines can be used. Techniques for soft agar growth or colony formation in suspension assays are described in Freshney, Culture of Animal Cells a Manual of Basic Technique, 3^{rd} ed., Wiley-Liss, New York (1994), herein incorporated by reference. See also, the methods section of Garkavtsev et al. (1996), supra, herein incorporated by reference.

Contact Inhibition and Density Limitation of Growth

Normal cells typically grow in a flat and organized pattern in a petri dish until they touch other cells. When the cells touch one another, they are contact inhibited and stop growing. When cells are transformed, however, the cells are not contact inhibited and continue to grow to high densities in disorganized foci. Thus, the transformed cells grow to a higher saturation density than normal cells. This can be detected morphologically by the formation of a disoriented monolayer of cells or rounded cells in foci within the regular pattern of normal surrounding cells. Alternatively, labeling index with [³H]-thymidine at saturation density can be used to measure density limitation of growth. See Freshney (1994), supra. The transformed cells, when contacted with cellular proliferation modulators, regenerate a normal phenotype and become contact inhibited and would grow to a lower density.

Contact inhibition and density limitation of growth assays can be used to identify YY1 modulators which are capable of inhibiting abnormal proliferation and transformation in host cells. Typically, transformed host cells (e.g., cells that are not contact inhibited) are used in this assay. For example, RKO or HCT116 cell lines can be used. In this assay, labeling index with [³H]-thymidine at saturation density is a preferred method of measuring density limitation of growth. Transformed host cells are contacted with a potential YY1 modulator and are grown for 24 hours at saturation density in non-limiting medium conditions. The percentage of cells labeling with [³H]-thymidine is determined autoradiographically. See, Freshney (1994), supra. The host cells contacted with a YY1 modulator would give arise to a lower labeling index compared to control (e.g., transformed host cells transfected with a vector lacking an insert).

Growth Factor or Serum Dependence

Growth factor or serum dependence can be used as an assay to identify YY1 modulators. Transformed cells have a lower serum dependence than their normal counterparts (see, e.g., Temin, J. Natl. Cancer Insti. 37:167-175 (1966); Eagle et al., J. Exp. Med. 131:836-879 (1970)); Freshney, supra. This is in part due to release of various growth factors by the transformed cells. When transformed cells are contacted with a YY1 modulator, the cells would reacquire serum dependence and would release growth factors at a lower level.

Tumor Specific Markers Levels

Tumor cells release an increased amount of certain factors (hereinafter “tumor specific markers”) than their normal counterparts. For example, plasminogen activator (PA) is released from human glioma at a higher level than from normal brain cells (see, e.g., Gullino, Angiogenesis, tumor vascularization, and potential interference with tumor growth. In Mihich (ed.): “Biological Responses in Cancer.” New York, Academic Press, pp. 178-184 (1985)). Similarly, tumor angiogenesis factor (TAF) is released at a higher level in tumor cells than their normal counterparts. See, e.g., Folkman, Angiogenesis and cancer, Sem Cancer Biol. (1992)). Other exemplified tumor specific markers include growth factors and cytokines.

Tumor specific markers can be assayed to identify YY1 modulators which decrease the level of release of these markers from host cells. Typically, transformed or tumorigenic host cells are used. Various techniques which measure the release of these factors are described in Freshney (1994), supra. Also, see, Unkless et al., J. Biol. Chem. 249:4295-4305 (1974); Strickland & Beers, J. Biol. Chem. 251:5694-5702 (1976); Whur et al., Br. J. Cancer 42:305-312 (1980); Gulino, Angiogenesis, tumor vascularization, and potential interference with tumor growth. In Mihich, E. (ed): “Biological Responses in Cancer.” New York, Plenum (1985); Freshney Anticancer Res. 5:111-130 (1985).

Invasiveness into Matrigel

The degree of invasiveness into Matrigel or some other extracellular matrix constituent can be used as an assay to identify YY1 modulators which are capable of inhibiting abnormal cell proliferation and tumor growth. Tumor cells exhibit a good correlation between malignancy and invasiveness of cells into Matrigel or some other extracellular matrix constituent. In this assay, tumorigenic cells are typically used as host cells. Therefore, YY1 modulators can be identified by measuring changes in the level of invasiveness between the host cells before and after the introduction of potential modulators. If a compound modulates YY1, its expression in tumorigenic host cells would affect invasiveness.

Techniques described in Freshney (1994), supra, can be used. Briefly, the level of invasion of host cells can be measured by using filters coated with Matrigel or some other extracellular matrix constituent. Penetration into the gel, or through to the distal side of the filter, is rated as invasiveness, and rated histologically by number of cells and distance moved, or by prelabeling the cells with ¹²⁵I and counting the radioactivity on the distal side of the filter or bottom of the dish. See, e.g., Freshney (1984), supra.

G₀/G₁ Cell Cycle Arrest Analysis

G₀/G₁ cell cycle arrest can be used as an assay to identify YY1 modulators. In this assay, cell lines, such as RKO or HCT116, can be used to screen YY1 modulators. The cells can be co-transfected with a construct comprising a marker gene, such as a gene that encodes green fluorescent protein, or a cell tracker dye. Methods known in the art can be used to measure the degree of G₁ cell cycle arrest. For example, a propidium iodide signal can be used as a measure for DNA content to determine cell cycle profiles on a flow cytometer. The percent of the cells in each cell cycle can be calculated. Cells contacted with a YY1 modulator would exhibit, e.g., a higher number of cells that are arrested in G₀/G₁ phase compared to control.

Tumor Growth In Vivo

Effects of YY1 modulators on cell growth can be tested in transgenic or immune-suppressed mice. Knock-out transgenic mice can be made, in which the endogenous YY1 gene is disrupted. Such knock-out mice can be used to study effects of YY1, e.g., as a cancer model, as a means of assaying in vivo for compounds that modulate YY1, and to test the effects of restoring a wild-type or mutant YY1 to a knock-out mouse.

Knock-out cells and transgenic mice can be made by insertion of a marker gene or other heterologous gene into the endogenous YY1 gene site in the mouse genome via homologous recombination. Such mice can also be made by substituting the endogenous YY1 with a mutated version of YY1, or by mutating the endogenous YY1, e.g., by exposure to carcinogens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987). These knock-out mice can be used as hosts to test the effects of various YY1 modulators on cell growth.

Alternatively, various immune-suppressed or immune-deficient host animals can be used. For example, genetically athymic “nude” mouse (see, e.g., Giovanella et al., J. Natl. Cancer Inst. 52:921 (1974)), a SCID mouse, a thymectomized mouse, or an irradiated mouse (see, e.g., Bradley et al., Br. J. Cancer 38:263 (1978); Selby et al., Br. J. Cancer 41:52 (1980)) can be used as a host. Transplantable tumor cells (typically about 10⁶ cells) injected into isogenic hosts will produce invasive tumors in a high proportions of cases, while normal cells of similar origin will not. Hosts are treated with YY1 modulators, e.g., by injection, optionally in combination with other cancer therapeutic agents, including chemotherapy, radiotherapy, immunotherapy or hormonal therapy. After a suitable length of time, preferably 4-8 weeks, tumor growth is measured (e.g., by volume or by its two largest dimensions) and compared to the control. Tumors that have statistically significant reduction (using, e.g., Student's T test) are said to have inhibited growth. Using reduction of tumor size as an assay, YY1 modulators which are capable, e.g., of inhibiting abnormal cell proliferation or sensitizing tumor cells to cancer therapies, can be identified.

In immune-suppressed or immune-deficient host animals, the inoculating tumor cells preferably overexpress or underexpress YY1. The inoculating tumor cells are also preferably resistant to conventionally used cancer therapies. Exemplified modulators include siRNA, NO donors, NF-κB inhibitors (i.e., dehydroxymethylepoxyquinomicin (DHMEQ)), proteasome inhibitors (i.e., Bortezomib, Velcade), and microtubule inhibitors (i.e., 2-Methoxyestradiol (2ME2) and vincristine). In one example, tumor cells resistant to death receptor-induced (e.g., DR5) apoptosis are inoculated as xenografts in SCID mice. The mice are subsequently treated with one or more inhibitors of YY1 (siRNA, NO donors, NF-κB inhibitors, etc.) combined with a death receptor agonist (e.g., a monoclonal antibody to DR5 or TRAIL).

Murine, rodent and other animal tumor models for studying cancer are generally described, for example, in Immunodeficient Animals: Models for Cancer Research, Arnold, et al., eds., 1996, S Karger Pub; Tumor Models in Cancer Research, Teicher, ed., 2002, Human Press; and Mouse Models of Cancer, Holland, ed., 2004, John Wiley & Sons. Specific murine tumor models for several different cancers have been described, including for example, metastatic colon cancer (Luo, et al., Cancer Cell (2004) 6:297), breast cancer (Rahman & Sarkar, Cancer Res (2005) 65:364), cholangiocarcinoma (Chen, et al., World J Gastroenterol (2005) 11:726), and prostate cancer (Tsingotjidou, et al., Anticancer Res (2001) 21:971 and U.S. Pat. No. 6,107,540).

### Screening Methods

The present invention also provides methods of identifying compounds that inhibit cancer growth or progression, for example, by inhibiting the binding of a YY1 protein to a YY1 binding protein or a nucleic acid. The compounds find use in inhibiting the growth of and promoting the regression of a tumor that overexpresses YY1 protein, for example, prostate cancer, ovarian cancer, lung cancer, renal cancer, breast cancer, colon cancer, leukemias, B-cell lymphomas (e.g., non-Hodgkin's lymphomas, including Burkitt's, Small Cell, and Large Cell lymphomas), hepatocarcinoma or multiple myeloma. The identified compounds can inhibit cancer growth or progression alone, or when used in combination with other cancer therapies, including chemotherapies, radiation therapies, hormonal therapies and immunotherapies.

Using the assays described herein, one can identify lead compounds that are suitable for further testing to identify those that are therapeutically effective modulating agents by screening a variety of compounds and mixtures of compounds for their ability to decrease or inhibit the binding of a YY1 protein to a YY1 binding protein or to DNA and inhibit the transcriptional regulation of gene products under YY1 repression. One particularly useful assay system utilized a reporter system where a reporter gene (i.e., luciferase or GFP) is operably linked to a promoter sequence comprising a YY1 binding sequence. Compounds of interest can be either synthetic or naturally occurring.

Screening assays can be carried out in vitro or in vivo. Typically, initial screening assays are carried out in vitro, and can be confirmed in vivo using cell based assays or animal models. For instance, proteins of the regenerating gene family are involved with cell proliferation. Therefore, compounds that inhibit the binding of a YY1 protein to a YY1 binding protein or nucleic acid can inhibit cell proliferation resulting from this binding interaction in comparison to cells unexposed to a test compound. Also, the binding of a YY1 protein to a YY1 binding protein or nucleic acid is involved with tissue injury responses, inflammation, and dysplasia. In animal models, compounds that inhibit the binding of a YY1 protein to a YY1 binding protein or nucleic acid can, for example, inhibit wound healing or the progression of dysplasia in comparison to an animal unexposed to a test compound. See, for example, Zhang, et al., World J Gastroenter (2003) 9:2635-41.

Usually, a compound that inhibits the binding of a YY1 protein to a YY1 binding protein or nucleic acid is synthetic. The screening methods are designed to screen large chemical or polymer (i.e., inhibitory RNA, including siRNA and antisense RNA, peptides, small organic molecules, etc.) libraries by automating the assay steps and providing compounds from any convenient source to the assays, which are typically run in parallel (e.g., in microtiter formats on microtiter plates in robotic assays).

The invention provides in vitro assays for inhibiting YY1 protein binding to a YY1 binding protein or nucleic acid in a high throughput format. For each of the assay formats described, “no modulator” control reactions which do not include a modulator provide a background level of YY1 binding interaction to a YY1 binding protein or nucleic acid. In the high throughput assays of the invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (96) modulators. If 1536 well plates are used, then a single plate can easily assay from about 100- about 1500 different compounds. It is possible to assay many different plates per day; assay screens for up to about 6,000-20,000, and even up to about 100,000-1,000,000 different compounds is possible using the integrated systems of the invention. The steps of labeling, addition of reagents, fluid changes, and detection are compatible with full automation, for instance using programmable robotic systems or “integrated systems” commercially available, for example, through BioTX Automation, Conroe, Tex.; Qiagen, Valencia, Calif.; Beckman Coulter, Fullerton, Calif.; and Caliper Life Sciences, Hopkinton, Mass.

Essentially, any chemical compound can be tested as a potential inhibitor of YY1 binding to a YY1 binding protein or nucleic acid for use in the methods of the invention. Most preferred are generally compounds that can be dissolved in aqueous or organic (especially DMSO-based) solutions are used. It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland), as well as providers of small organic molecule and peptide libraries ready for screening, including Chembridge Corp. (San Diego, Calif.), Discovery Partners International (San Diego, Calif.), Triad Therapeutics (San Diego, Calif.), Nanosyn (Menlo Park, Calif.), Affymax (Palo Alto, Calif.), ComGenex (South San Francisco, Calif.), and Tripos, Inc. (St. Louis, Mo.).

Compounds also include those that can regulate YY1 transcription and post-transcriptional processing and compounds that can regulate gene expression under the control of YY1. Reporter systems can be used for this analysis.

In one preferred embodiment, inhibitors of YY1 protein binding to a YY1 binding protein or nucleic acid are identified by screening a combinatorial library containing a large number of potential therapeutic compounds (potential modulator compounds). Such “combinatorial chemical or peptide libraries” can be screened in one or more assays, as described herein, to identify those library members particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art (see, for example, Beeler, et al., Curr Opin Chem. Biol. 9:277 (2005) and Shang and Tan, Curr Opin Chem. Biol. 9:248 (2005). Libraries of use in the present invention can be composed of amino acid compounds, nucleic acid compounds, carbohydrates or small organic compounds. Carbohydrate libraries have been described in, for example, Liang et al., Science, 274:1520-1522 (1996) and U.S. Pat. No. 5,593,853.

Representative amino acid compound libraries include, but are not limited to, peptide libraries (see, e.g., U.S. Pat. Nos. 5,010,175; 6,828,422 and 6,844,161; Furka, Int. J. Pept. Prot. Res. 37:487-493 (1991); Houghton et al., Nature 354:84-88 (1991) and Eichler, Comb Chem High Throughput Screen. 8:135 (2005)), peptoids (PCT Publication No. WO 91/19735), encoded peptides (PCT Publication WO 93/20242), random bio-oligomers (PCT Publication No. WO 92/00091), vinylogous polypeptides (Hagihara et al., J. Amer. Chem. Soc. 114:6568 (1992)), nonpeptidal peptidomimetics with β-D-glucose scaffolding (Hirschmann et al., J. Amer. Chem. Soc. 114:9217-9218 (1992)), peptide nucleic acid libraries (see, e.g., U.S. Pat. No. 5,539,083), antibody libraries (see, e.g., U.S. Pat. Nos. 6,635,424 and 6,555,310; PCT/US96/10287, and Vaughn et al., Nature Biotechnology, 14(3):309-314 (1996)), and peptidyl phosphonates (Campbell et al., J. Org. Chem. 59:658 (1994)).

Representative nucleic acid compound libraries include, but are not limited to, genomic DNA, cDNA, mRNA, inhibitory RNA (RNAi, siRNA) and antisense RNA libraries. See, Ausubel, Current Protocols in Molecular Biology, supra, and Sambrook and Russell, Molecular Cloning: A Laboratory Manual, 2000, Cold Spring Harbor Laboratory Press. Nucleic acid libraries are described in, for example, U.S. Pat. Nos. 6,706,477; 6,582,914; and 6,573,098. cDNA libraries are described in, for example, U.S. Pat. Nos. 6,846,655; 6,841,347; 6,828,098; 6,808,906; 6,623,965; and 6,509,175. RNA libraries, for example, ribozyme, RNA interference or siRNA libraries, are reviewed in, for example, Downward, Cell 121:813 (2005) and Akashi, et al., Nat Rev Mol Cell Biol. 6:413 (2005). Antisense RNA libraries are described in, for example, U.S. Pat. Nos. 6,586,180 and 6,518,017.

Representative small organic molecule libraries include, but are not limited to, diversomers such as hydantoins, benzodiazepines and dipeptides (Hobbs et al., Proc. Nat. Acad. Sci. USA 90:6909-6913 (1993)), analogous organic syntheses of small compound libraries (Chen et al., J. Amer. Chem. Soc. 116:2661 (1994)), oligocarbamates (Cho et al., Science 261:1303 (1993)); benzodiazepines (U.S. Pat. No. 5,288,514; and Baum, C&EN, Jan 18, page 33 (1993)); isoprenoids (e.g., U.S. Pat. No. 5,569,588); thiazolidinones and metathiazanones (e.g., U.S. Pat. No. 5,549,974); pyrrolidines (e.g., U.S. Pat. Nos. 5,525,735 and 5,519,134); morpholino compounds (e.g., U.S. Pat. No. 5,506,337); tetracyclic benzimidazoles (e.g., U.S. Pat. No. 6,515,122); dihydrobenzpyrans (e.g., 6,790,965); amines (e.g., U.S. Pat. No. 6,750,344); phenyl compounds (e.g., 6,740,712); azoles, (e.g., 6,683,191); pyridine carboxamides or sulfonamides (e.g., 6,677,452); 2-aminobenzoxazoles (e.g., U.S. Pat. No. 6,660,858); isoindoles, isooxyindoles, or isooxyquinolines (e.g., 6,667,406); oxazolidinones (e.g., U.S. Pat. No. 6,562,844); and hydroxylamines (e.g., U.S. Pat. No. 6,541,276).

Of particular interest are libraries of nitric oxide donor compounds, for example, libraries of molecules with core structures like the nitric oxide donor compounds disclosed in U.S. Pat. Nos. 6,897,218; 6,897,194; 6,780,849; 6,642,260; 6,538,033; 6,451,337; and 5,698,738 (see also, Balogh, et al., Comb Chem High Throughput Screen. 8:347 (2005)). Libraries of nitric oxide compounds have been developed by Nitromed of Lexington, Mass.

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.).

### Administration and Pharmaceutical Compositions

Molecules and compounds identified that modulate the expression and/or function of YY1 are useful in treating cancers that overexpress YY1. YY1 modulators can be administered alone or co-administered in combination with conventional chemotherapy, radiotherapy or immunotherapy.

Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 20^{th} ed., 2003, supra).

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

Preferred pharmaceutical preparations deliver one or more YY1 inhibitors, optionally in combination with one or more chemotherapeutic agents, in a sustained release formulation. Typically, the YY1 inhibitor is administered therapeutically as a sensitizing agent that increases the susceptibility of tumor cells to other cytotoxic cancer therapies, including chemotherapy, radiation therapy, immunotherapy and hormonal therapy. In some embodiments, the YY1 inhibitor can be an NO donor, including those listed supra, a conjugate comprising NO and another agent (i.e., NO conjugated to aspirin), or an activator of inducible nitric oxide synthase.

In therapeutic use for the treatment of cancer, the compounds utilized in the pharmaceutical method of the invention are administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound being employed. For example, dosages can be empirically determined considering the type and stage of cancer diagnosed in a particular patient. The dose administered to a patient, in the context of the present invention should be sufficient to effect a beneficial therapeutic response in the patient over time. The size of the dose also will be determined by the existence, nature, and extent of any adverse side-effects that accompany the administration of a particular vector, or transduced cell type in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The pharmaceutical preparations are typically delivered to a mammal, including humans and non-human mammals. Non-human mammals treated using the present methods include domesticated animals (i.e., canine, feline, murine, rodentia, and lagomorpha) and agricultural animals (bovine, equine, ovine, porcine).

### Diagnostic Methods

The present invention also provides methods of diagnosing a cancer, including wild-type, truncated or alternatively spliced forms of YY1. Diagnosis can involve determining the level of YY1 expression (transcription or translation), YY1 DNA binding activity or YY1 intracellular localization in a patient and then comparing the level to a baseline or range. Typically, the baseline value is representative YY1 expression levels, YY1 DNA binding activity or YY1 intracellular localization in a healthy person not suffering from cancer. Variation of levels of a polypeptide or polynucleotide of the invention from the baseline range (either up or down) indicates that the patient has a cancer or is at risk of developing a cancer. In some embodiments, the level of YY1 expression, YY1 DNA binding activity or YY1 intracellular localization are measured by taking a blood, urine or tissue sample from a patient and measuring the amount of a polypeptide or polynucleotide of the invention in the sample using any number of detection methods, such as those discussed herein.

Antibodies can be used in assays to detect differential protein expression and protein localization in patient samples, e.g., ELISA assays, immunoprecipitation assays, and immunohistochemical assays. In one embodiment, tumor tissue samples are used in immunohistochemical assays and scored according to standard methods known in the art. PCR assays can be used to detect expression levels of nucleic acids, as well as to discriminate between variants in genomic structure, such as insertion/deletion mutations, truncations or splice variants. Immunohistochemistry and/or immunofluorescence techniques can be used to detect increased nuclear localization of YY1 proteins.

In some embodiments, overexpression of YY1 in a cancerous or potentially cancerous tissue in a patient may be diagnosed or otherwise evaluated by visualizing expression levels and localization in situ of a YY1 polynucleotide, a YY1 polypeptide, or fragments of either. Those skilled in the art of visualizing the presence or expression of molecules including nucleic acids, polypeptides and other biochemicals in the tissues of living patients will appreciate that the gene expression information described herein may be utilized in the context of a variety of visualization methods. Such methods include, but are not limited to, single-photon emission-computed tomography (SPECT) and positron-emitting tomography (PET) methods. See, e.g., Vassaux and Groot-wassink, “In Vivo Noninvasive hnaging for Gene Therapy,” J. Biomedicine and Biotechnology, 2: 92-101 (2003).

PET and SPECT imaging shows the chemical functioning of organs and tissues, while other imaging techniques—such as X-ray, CT and MRI—show structure. The use of PET and SPECT imaging is useful for qualifying and monitoring the development of cancers that overexpress YY1 and/or therapy resistant cancers, including prostate cancer, ovarian cancer, lung cancer, renal cancer, breast cancer, colon cancer, leukemias, B-cell lymphomas, myelomas and hepatocarcinomas. In some instances, the use of PET or SPECT imaging allows diseases to be detected years earlier than the onset of symptoms. The use of small molecules for labelling and visualizing the presence or expression of polypeptides and nucleotides has had success, for example, in visualizing proteins in the brains of Alzheimer's patients, as described by, e.g., Herholz K et al., Mol Imaging Biol., 6(4):239-69 (2004); Nordberg A, Lancet Neurol., 3(9):519-27 (2004); Neuropsychol Rev., Zakzanis K K et al., 13(1):1-18 (2003); Kung M P et al, Brain Res., 1025(1-2):98-105 (2004); and Herholz K, Ann Nucl Med., 17(2):79-89 (2003).

A YY1 polypeptide, a YY1 polynucleotide, or fragments of either, can be used in the context of PET and SPECT imaging applications. After modification with appropriate tracer residues for PET or SPECT applications, molecules which interact or bind with a YY1 transcript or with any polypeptides encoded by those transcripts may be used to visualize the patterns of gene expression and facilitate diagnosis of cancers that overexpress YY1.

### Compositions, Kits and Integrated Systems

The invention provides compositions, kits and integrated systems for practicing the assays described herein using polypeptides or polynucleotides of the invention, antibodies specific for polypeptides or polynucleotides of the invention, etc.

The invention provides assay compositions for use in solid phase assays; such compositions can include, for example, one or more polynucleotides or polypeptides of the invention immobilized on a solid support, and a labeling reagent. In each case, the assay compositions can also include additional reagents that are desirable for hybridization. Modulators of expression or activity of polynucleotides or polypeptides of the invention can also be included in the assay compositions.

The invention also provides kits for carrying out the therapeutic and diagnostic assays of the invention. The kits typically include one or more probes that comprises an antibody or nucleic acid sequence that specifically binds to polypeptides or polynucleotides of the invention, and a label for detecting the presence of the probe. The kits can find use, for example for measuring the levels of YY1 protein or YY1 transcripts, or for measuring YY1 DNA-binding activity. The kits may include several polynucleotide sequences encoding polypeptides of the invention. Kits can include any of the compositions noted above, and optionally further include additional components such as instructions to practice a high-throughput method of assaying for an effect on expression of the genes encoding the polypeptides of the invention, or on activity of the polypeptides of the invention, one or more containers or compartments (e.g., to hold the probe, labels, or the like), a control modulator of the expression or activity of polypeptides of the invention, a robotic armature for mixing kit components or the like.

The invention also provides integrated systems for high-throughput screening of potential modulators for an effect on the expression or activity of the polypeptides of the invention. The systems typically include a robotic armature which transfers fluid from a source to a destination, a controller which controls the robotic armature, a label detector, a data storage unit which records label detection, and an assay component such as a microtiter dish comprising a well having a reaction mixture or a substrate comprising a fixed nucleic acid or immobilization moiety.A number of robotic fluid transfer systems are available, or can easily be made from existing components. For example, a Zymate XP (Zymark Corporation; Hopkinton, Mass.) automated robot using a Microlab 2200 (Hamilton; Reno, Nev.) pipetting station can be used to transfer parallel samples to 96 well microtiter plates to set up several parallel simultaneous STAT binding assays.

Optical images viewed (and, optionally, recorded) by a camera or other recording device (e.g., a photodiode and data storage device) are optionally further processed in any of the embodiments herein, e.g., by digitizing the image and storing and analyzing the image on a computer. A variety of commercially available peripheral equipment and software is available for digitizing, storing and analyzing a digitized video or digitized optical image, e.g., using PC (Intel x86 or Pentium chip-compatible DOS®, OS2® WINDOWS®, WINDOWS NT®, WINDOWS95®, WINDOWS98®, or WINDOWS2000® based computers), MACINTOSH®, or UNIX® based (e.g., SUN® work station) computers.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. The apparatus and methods of the invention are easily used for viewing any sample, e.g., by fluorescent or dark field microscopic techniques.

### EXAMPLES

The following examples are offered to illustrate, but not to limit the claimed invention.

### Example 1

### Overexpression of YY1 in Prostate Cancer Cells

### Materials and Methods

### Cell Culture and Reagents

The human androgen-independent PC-3 cell line was originally obtained from the American Type Culture Collection (ATCC, Manassas, Va.). The cell were cultured in RPMI 1640 medium with 10% beat inactivated FBS with antibiotics and the culture was maintained as monolayer on plastic dish and incubated at 37 degrees at a 5% CO2 incubator. The rabbit anti-YY1 antibody and the YY1 peptide were obtained from Geneka, (Montreal, Canada). The antibody was titrated for optimal concentration to be used for both Western and immunohistochemistry. The specificity of the antibody was demonstrated by neutralizing the activity with the YY1 peptide used for immunization. In addition, normal rabbit IgG had no activity (Vector, Burlingame, Calif.). The I actin antibody used in the Western blot was purchased from Chemicon (Temecula, Calif.).

### Western Blot Analysis

PC-3 cells were cultured at a low serum concentration (0.1%) 18 h prior to each treatment. After incubation, the cells were maintained in serum free medium (control), or treated with TNF-α, (1, 10, and 100 U/ml-24 h). The cells were then lysed at 4° C. in RIPA buffer {50 mM Tris-HCl (pH 7.4), 1% Nonidet P-40, 0.25% sodium deoxycholate, 150 mM NaC1}, and supplemented with one tablet of protease inhibitor cocktail, Complete Mini Roche (Indianapolis, Ind.). Protein concentration was determined by a DC protein assay kit (Bio-Rad, Hercules, Calif.). An aliquot of total protein lysate was diluted in an equal volume of 2×SDS sample buffer 6.2 mM Tris (pH6.8), 2.3% SDS, 5% mecraptoethanoel, 10% glycerol, and 0.02% bromphenol blue and boiled for 10 minutes. The cell lysates (40 were then electrophoresed on 12% SDS-PAGE gels (Bio-Rad) and were subjected to Western blot analysis as previously reported (Jazirehi, A. R. et al. Clin. Cancer Res., 7:3874-83 (2001)). Levels of f were used to normalize the YY1 expression. Relative concentrations were assessed by densitometric analysis of digitized autographic images, performed on a Macintosh computer (Apple Computer Inc., Cupertino, Calif.) using the public domain NIH Image J Program (available on the internet at http://rsb.info.nih.gov/ij/).

### Prostate Tissue Microarray (TMA)

Formalin-fixed paraffin-embedded prostate tissue samples were provided through the Department of Pathology at the UCLA Medical Center under IRB approval. Primary radical prostatectomy cases from 1984-1995 were randomly selected from the pathology database. The original H&E stained slides were reviewed by a study pathologists (D.S.) utilizing the Gleason histological grading (Gleason, D. F. Cancer Chemother. Rep., 50:125-8 (1996)) and the 1997 AJCC/UICC TNM classification systems (Sobin, L. H. et al. Cancer, 80:1803-4 (1997)).

TMAs were constructed as described (Kononen, J. et al. Nat. Med., 4:844-7 (1998)). At least 3 replicate tumor samples were taken from donor tissue blocks in a widely representative fashion. Tumor samples were accompanied by matching benign (morpho logically normal or hypertrophic) and in situ neoplastic lesions, (PIN), when available.

### Patient Group and Database

A retrospective analysis for outcome assessment was based on detailed anonymized clinicopathólogic information linked to the TMA tissue specimens. Data sources included the original surgical pathology reports, the pathology case review, the Tumor Registry of the UCLA Cancer Program of the Jonsson Comprehensive Cancer Center and the UCLA Department of Urology.

Case material from 246 prostatectomies was arrayed into 3 blocks encompassing a total of 1,364 individual tissue cores. All cases were of the histological type adenocarcinoma, conventional, not otherwise specified (Young, R H. et al. “In: Atlas of Tumor Pathology.” Series 3. Washington, D.C.: Armed Forces Institute of Pathology; (2000)). For clinical analyses, patients that were treated preoperatively with neoadjuvant hormones were excluded from the analysis (n=20). Another 23 cases were not evaluable predominantly due to a lack of target tumor tissue, and 12 remaining cases bad missing outcomes data. Therefore, of 246 total cases, 190 (77%) were available for outcome studies, and each case was represented by an average of 3.2 informative tumor spots. Tissue spots from all 246 cases were included in the histological spot-level distribution analyses of YY1 staining, and 79% of these tissue spots were informative.

Table 1 shows the clinicopathologic data for the 190 patients included in the outcomes analysis. The median age at the time of surgery was 65 (range 46 to 76). 112 (59%) patients were low grade (Gleason score 2-6); 78 (41%) were high grade (Gleason score 7-10). 124 cases (65%) belong to the AJCC 1997 stage grouping II; 57 (30%) into grouping III and 9 (5%) into grouping IV. The majority of tumors (n=115, 64%) were confined to the prostate (organ confined=T2a and T2b with negative lymph nodes and no capsular extension). 128 (67%) patients were margin negative, 62 (33%) margin positive, 32 (17%) had seminal vesicle invasion. Regarding capsular invasion, 44 (23%) had no invasion, 107 (56%) had invasion, and 39 (21%) had capsular extension. Concurrent regional lymphadenectomy accompanied 187 (98%) cases. The maximum pre-operative serum PSA was known for 169 patients (89%), with a median log value of 2.2, (range 0.3-4.3).

The median follow-up time for all patients was 84 months (range 0-182). Recurrence, defined as a postoperative serum PSA of 0.2 ng/ml or greater, was seen in 65 (34%) patients. For the recurring group, time to recurrence, defined as the interval from the date of diagnosis to PSA recurrence, had a median of 21 months (range 1.0-115). The median follow-up time for the recurring and non-recurring groups was 98 (4-182) and 72 months (range 0-163), respectively. 160 (84%) patients were alive at last contact, and their median time of follow-up was 84 months (range 2-182). Of the 30 patients who were dead at last contact, only 9 (30% of dead) were disease-related deaths. Of those who died of disease, the median survival time was 68 months (range 21-143).

### Immunohistochemistry

A standard 2-step indirect avidin-biotin complex (ABC) method was used (Vector Laboratories, Burlingame, Calif.). Tissue array sections (4 μm-thick) were cut immediately prior to staining using the TMA sectioning aid (Instrumedics, N.J.). Following deparaffinization in xylene, the sections were rehydrated in graded alcohols and endogenous peroxidase was quenched with 3% hydrogen peroxide in methanol at room temperature. The sections were placed in 95° C. solution of 0.01 M sodium citrate buffer (pH 6.0) for antigen retrieval, and then blocked with 5% normal goat serum for 30 min. Endogenous biotin was blocked with sequential application of avidin D then biotin (A/B blocking system, Vector Laboratories, Burlingame, Calif.). Primary rabbit anti-human YY1 polyclonal IgG₁ antibody (Geneka Biotechnology, Inc., Montreal, Quebec, Canada) was applied at a 1:1000 dilution (0.2 ug/ml) for 60 min. at room temperature. After washing, biotinylated goat anti-rabbit IgG (Vector Laboratories, Burlingame, Calif.) was applied for 30 min at room temperature. The ABC complex was applied for 25 min. followed by the chromogen diaminobenzidine (DAB). 10 mM PBS at pH 7.4 was used for all wash steps and dilutions. Incubations were performed in a humidity chamber. The sections were counterstained with Harris' Hematoxylin, followed by dehydration and mounting.

Antibody specificity was tested by concentration-dependent inhibition of staining using the immunizing YY1 peptide (Geneka Biotechnology, Inc.). Anti-YY1 antibody was preincubated for 3 hours at room temperature with a 0×, 5×, or 10× molar excess of peptide. The antibody in the presence or absence of the peptide was then added to a mini prostate array (16 spots) and stained as described above.

### Scoring of Immunohistochemistry

Semi-quantitative assessment of antibody staining on the TMAs was performed by a study pathologist (A.R.) blinded to the clinicopathologic variables. The target tissue for scoring was the glandular prostatic epithelium, scoring of benign tissues did not include basal cells. Tissue spot histology and grading was confirmed on Hematoxylin and Eosin stained TMA slides, as well as on the counterstained study slides. The staining intensities of the nuclear and cytoplasmic cellular compartments were scored separately, each on a 0-3 scale (0=negative; 1=weak; 2=moderate; 3=strong staining). In addition, the frequency of positive target cells (range 0-100%) was scored for each TMA spot. To arrive at a summary expression score of each case for outcomes analysis, we pooled the tumor spot data by taking the median value of the staining frequency of all tumor spots. We then dichotomized the pooled summary expression, choosing a 50% staining frequency as a cut-off value for distinguishing “low” (n=15) from “high” (n=175) expressing cases. This was anatural cut-off value since only 9% of cases had a pooled positivity value between 25-75%, therefore only rare cases would be found at or near this cut-off point.

### Statistical Analysis

Associations between YY1 expression and clinicopathologic variables were tested using the Pearson chi-square test. To test whether the staining of YY1 differed between various histological groups, we used the Kruskal-Wallis test, which is a non-parametric multi-group comparison test. We used the Pearson correlations and corresponding p-values to study the relationship between nuclear and cytoplasmic staining intensities and frequencies. Recurrence was defined as a rising total PSA >0.2 ng/ml status post prostatectomy, and time to recurrence was calculated from the date of the primary surgery. Patients without recurrence at last follow-up were censored. Kaplan-Meier plots were used to visualize recurrence-free time distributions and the log rank test was used to test for differences between them To assess which covariates affect recurrence-free time, we fit both univariate and multivariate Cox proportional hazards models. For each covariate, we list the 2 sided p-value, the hazard ratio and its 95% confidence interval. A p-value smaller than 0.05 was accepted as significant. The proportional hazards assumption was checked through the use of Schoenfeld residuals. All analyses were conducted with the freely available software package R (http://www.r-project.org).

### Results

This study used tissue microarrays to investigate the expression and localization of YY1 in 246 hormone naïve prostate cancer patients who underwent radical prostatectomy. YY1 was immunohistochemically analyzed in 246 prostate cancer patients who underwent radical prostatectomy. YY1 nuclear and cytoplasmic protein expression was higher in intensity and in frequency in neoplastic compared to matched benign tissues (all p<0.0001). Nuclear and cytoplasmic YY1 expression in tumor samples of Gleason grade 1-2 displayed only insignificant increases compared to normal levels (p=0.355 and p=0.150, respectively), whereas the expression of YY1 was significantly elevated from normal in tumor samples of Gleason grades 3-5 (p<0.0001 for both nuclear and cytoplasmic staining), and in prostatic intraepithelial neoplasia (PIN), (p=0.0014 for nuclear, and p<0.0001 for cytoplasmic staining). Using Cox regression analysis, we found evidence that low nuclear YY1 staining is associated with shorter recurrence times for all patients (p=0.0327), as well as for patients with low Gleason scores (p=0.0263).

YY1 nuclear expression is predominantly elevated in early malignancy (PIN), as well as in tumors of intermediate to high morphologic grade. Hence, it has a role in both tumor initiation and disease progression. Loss of YY1 expression is associated with an increased risk of recurrence, suggesting a protective role in prostate cancer and other cancers. YY1 expression may facilitate identification of prostate and other cancer patients, including those presenting with low grade tumors, who have a higher risk of tumor recurrence, and therefore may benefit from more aggressive follow-up and treatment modalities.

### YY1 Expression in PC3

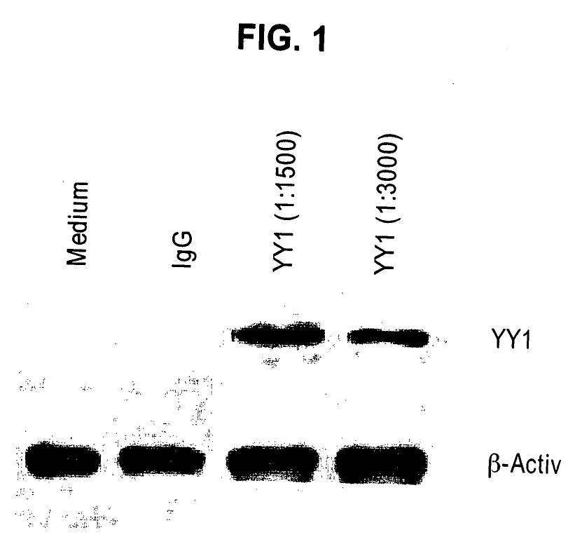

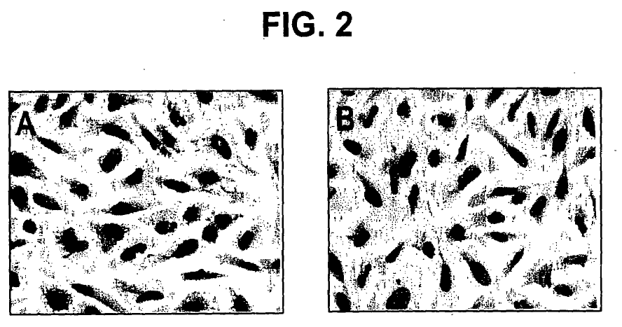

YY1 is a transcription factor that demonstrates context-specific repression or activation activity (Shi, Y. et al. Biochim. Biophys. Acta., 1332:F49-66 (1997)). Recently, we have demonstrated that nitric oxide indirectly up-regulates the expression of Fas by blocking the silencing effect of YY1 (Garban, H. J. et al. J. Immunol., 167:75-81 (2001)). The apparent role of YY1 in modulating Fas expression, combined with postulated role of TNF receptor family members in tumor progression and resistance (Thompson, C. B. Science, 267:1456-62 (1995); Igney, F. H. et al., Nat. Rev. Canc., 2:277-88 (2002)), prompted us to examine the expression distribution of YY1 in normal and malignant prostate tissue. We first examined the androgen-independent human prostate cancer cell line, PC3 for YY1 expression by Western blot analysis. Abundant expression was detected in these cells, yielding a prominent 68 kDa band (FIG. 1). Immunohistochemistry revealed that ≧95% of the cells expressed YY1, predominantly within the nucleus (FIG. 2). These findings establish the specificity of the anti-YY1 antibody for immunohistochemical analyses in prostate whole tissues and tissue nicroarrays.

### YY1 Expression in Prostate Tissue—Whole Tissue Sections

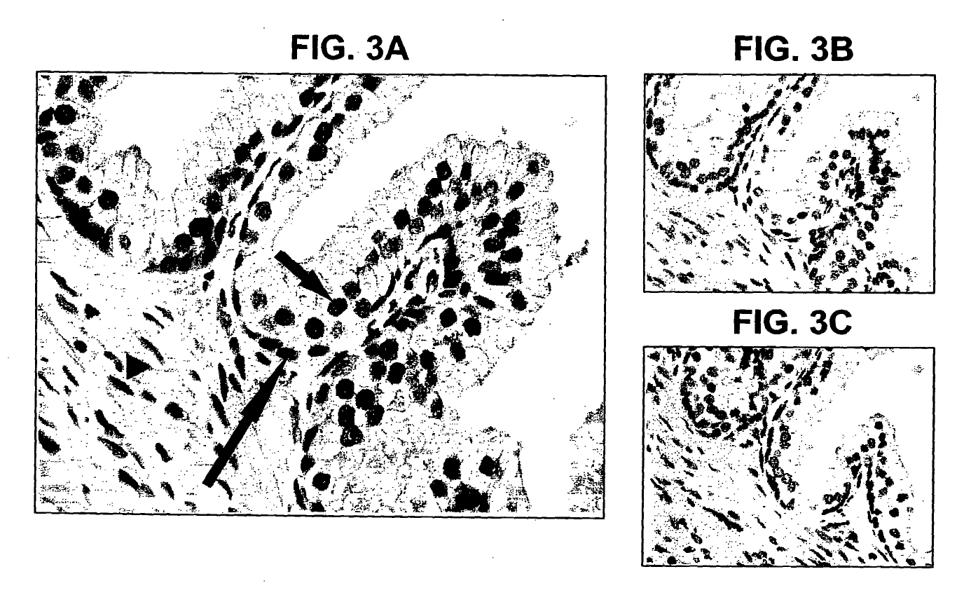

We further examined the expression of YY1 in morphologically normal/BPH (non malignant) human prostate tissue by immunohistochemistry on whole tissue sections. Consistently, staining was observed in the glandular epithelium, basal cells, and occasionally in stromal fibromuscular cells (FIG. 3A). Approximately 90% of the prostatic epithelium stained positive, typically with widely varying, but predominantly weak to moderate, intensities. Notably, the same predominantly nuclear expression pattern seen in the cell culture was also observed in these whole tissues (FIG. 3A). A lack of staining with non-immune sera, and a dose-dependent inhibition of staining through preincubation of the antibody with the immunogen peptide, confirmed that the staining observed was specific (FIGS. 3B and 3C, respectively).

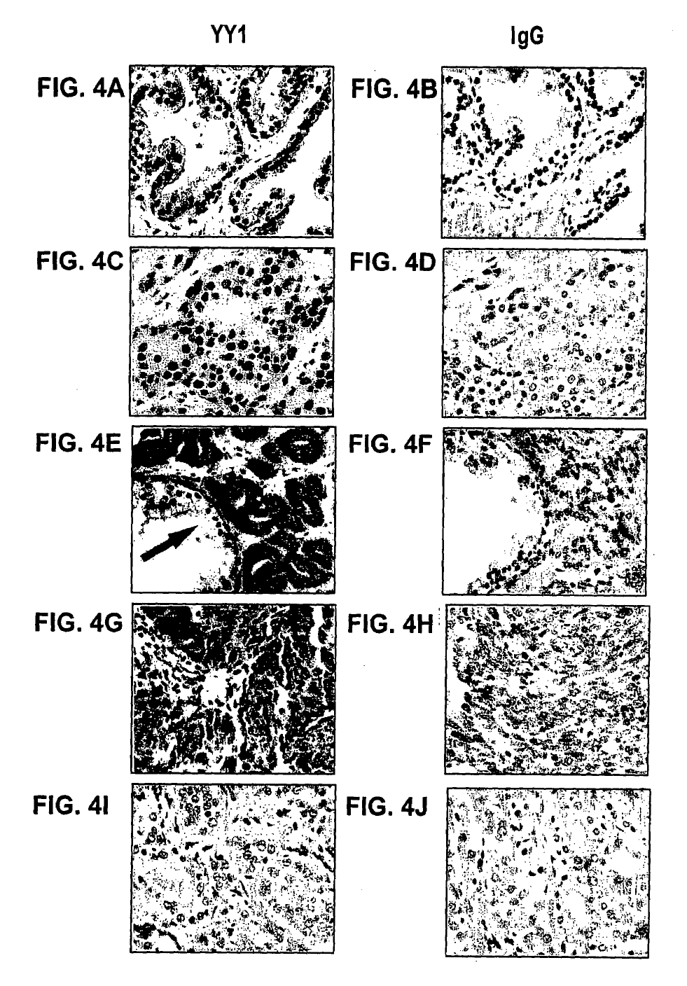

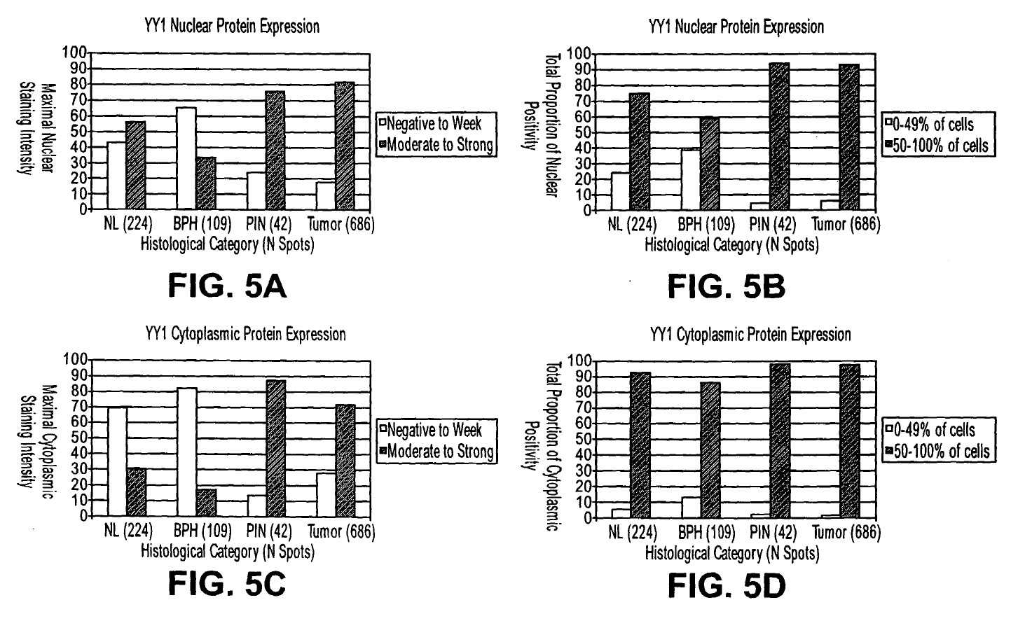

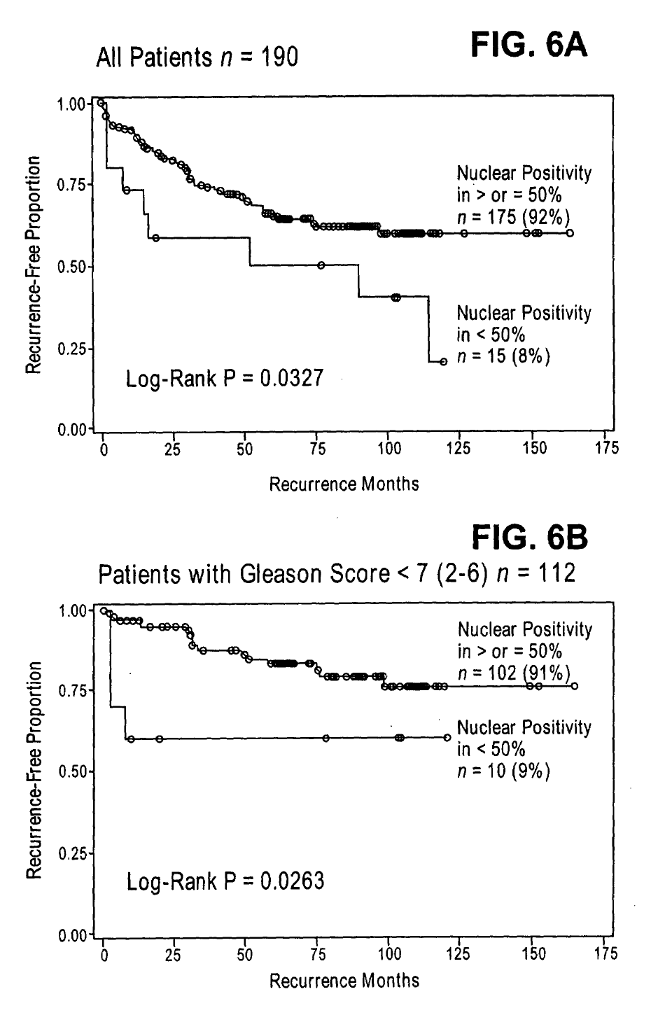

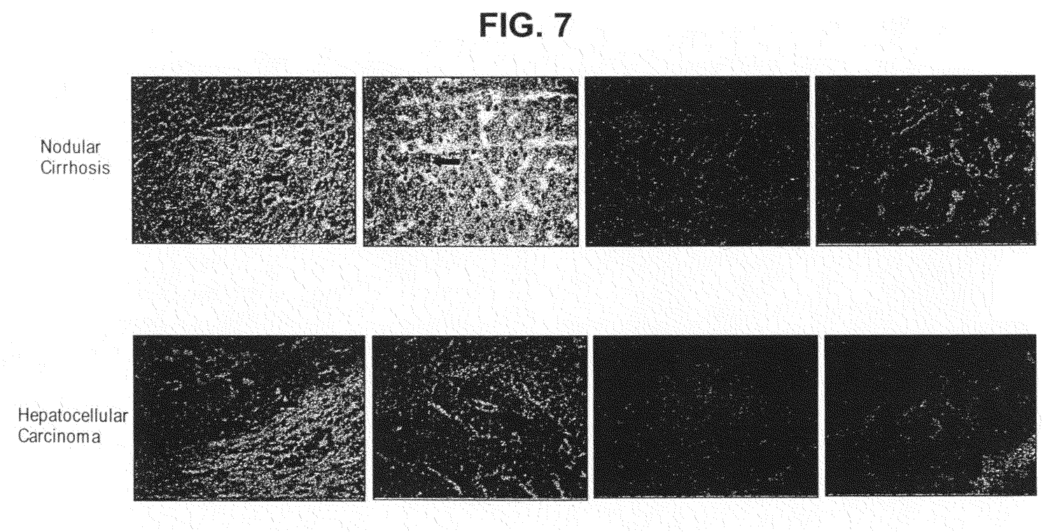

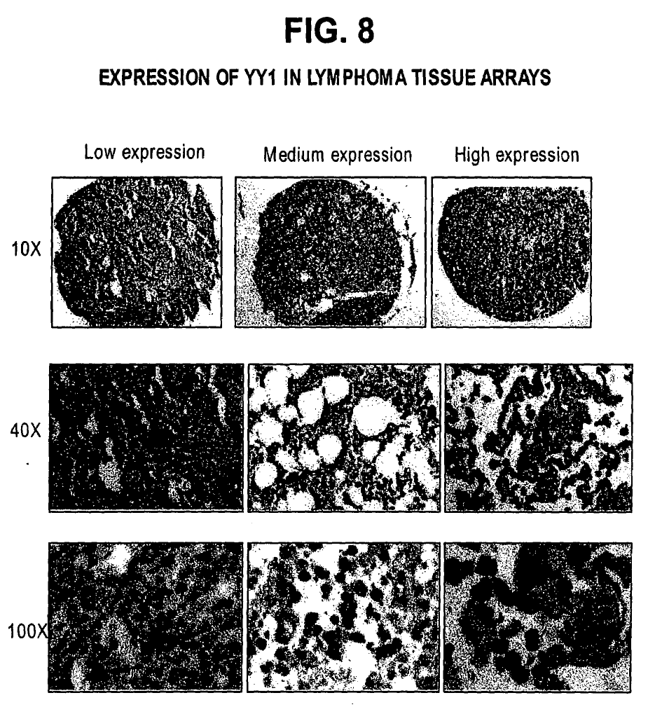

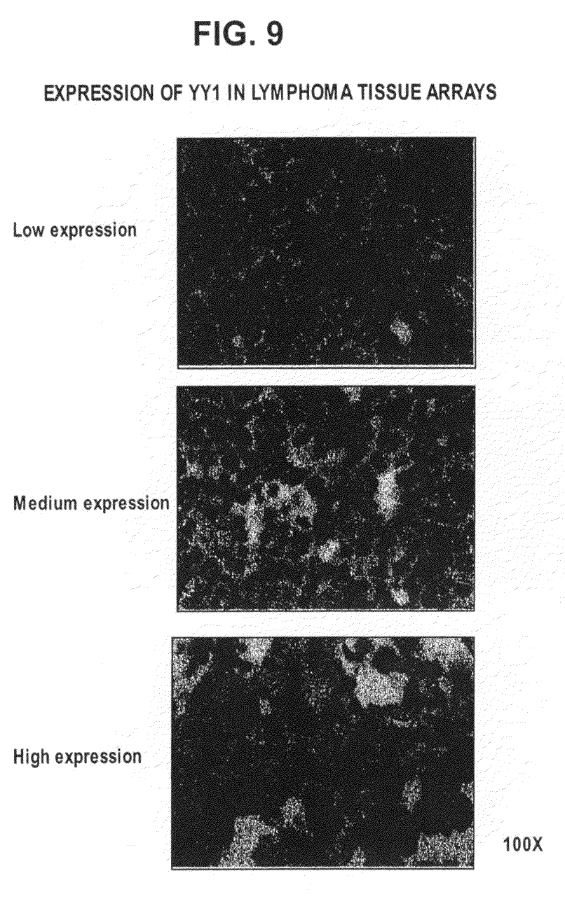

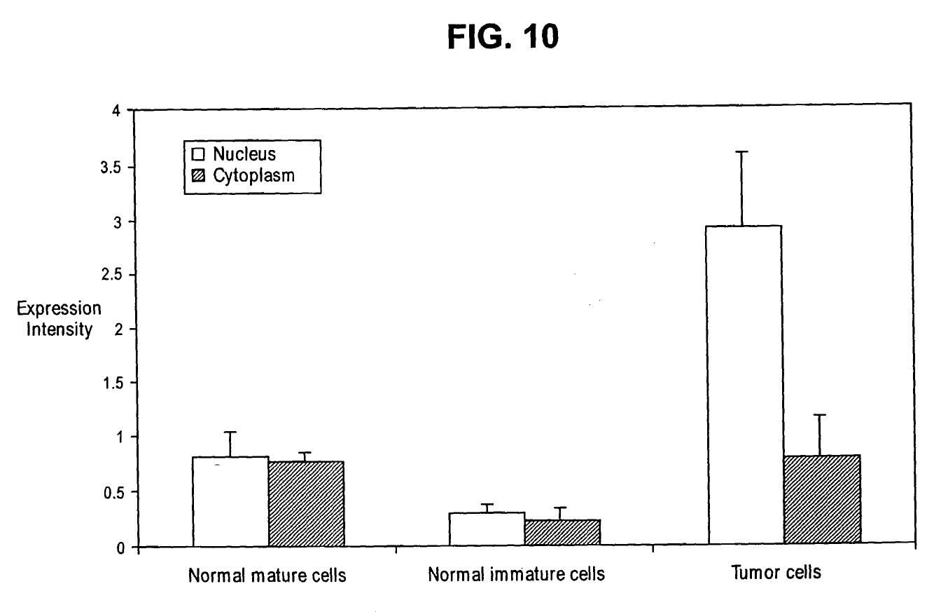

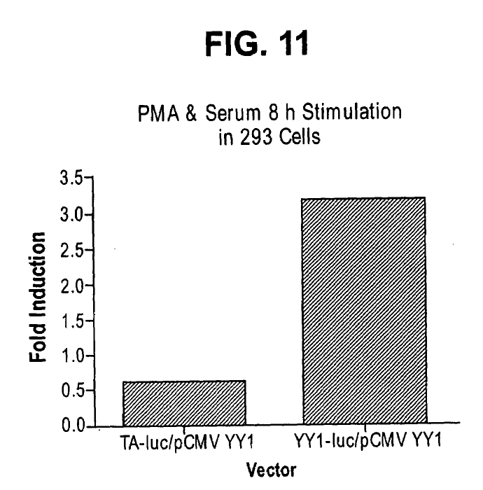

We next examined the expression of YY1 on whole tissue sections representing ten human prostate carcinomas. FIG. 4 shows representative patterns of staining that were observed. Compared to the typically pronounced nuclear staining seen in non-malignant epithelium (FIG. 4A), two low-grade tumors demonstrated weak or minimal YY1 staining (example in FIG. 4I) while another low-grade tumor exhibited strong nuclear staining and diffuse cytoplasmic staining (FIG. 4E). Two high-grade tumors were also examined; one demonstrated weak to moderate nuclear staining (FIG. 4C), while the other showed relatively strong nuclear and cytoplasmic staining (FIG. 4G). This complex set of staining patterns prompted us to examine a larger sample population using tissue microarray technology.

### YY1 Expression on a Prostate Tissue Microarray

We next evaluated the protein expression of YY1 among clinical prostate samples on the TMA platform to examine the prevalence or loss of YY1 expression among hormone naïve patients with predominantly early stage prostate cancer.

### Distributions of YY1 Expression

Each array spot was scored for YY1 protein expression by staining intensity and frequency, and cellular location (cytoplasmic and nuclear), with confirmation of histological category. We examined YY1 expression across histological categories on all 1061 informative primary site tissue spots (data for 12 lymph node metastases was not included; therefore 1073 (79%) of all spots were informative). Distribution graphs of nuclear YY1 staining intensity and frequency are seen in FIGS. 5A and 5B, respectively. An overall increase in YY1 staining in both tumor and PIN samples was noted over matched morphologically normal and BPH tissues (Table 2; p<0.0001). As a group, 82% of tumor-containing and 76% of PIN-containing spots showed moderate to strong nuclear staining, whereas normal and BPH tissues from the same case pool showed only 57% and 34% nuclear staining, respectively, at the same level (FIG. 5A). Interestingly, the proportion of tumor spots displaying moderate to strong staining increases abruptly with grade ≧Gleason grade 3 (graph not shown). Low Gleason grade histologies (U.S. Cancer Statistics Working Group. “United States Cancer Statistics: 1999 Incidence. Atlanta (GA): Department of Health and Human Services, Centers for Disease Control and Prevention and National Cancer Institute” (2002); Miniño, A. M. et al, “National Vital Statistics Reports. Centers for Disease Control and Prevention” Volume 50, Number 15 Sep. 16, 2002) stained at this level in 65% of spots (normal histology staining the same in 57% of spots), while grades 3, 4 and 5 stained at this intensity in 84%, 87% and 79% of spots, respectively (grade 1-2 versus grade ≧3, p<0.0001).

The distribution of YY1 staining as a proportion of positive cells in each histologic category (FIG. 5B) follows the same trend as was seen with intensity, a higher proportion of tumor and PIN target cells stain in the 50-100% category (p<0.0001). As a pooled group, 93% of tumor-containing and 95% of PIN-containing spots showed YY1 positive staining in ≧50% of the appropriate target cells, whereas normal and BPH tissues from the same case pool showed 76% and 61% nuclear staining, respectively, at the same frequency range. Therefore, in the neoplastic lesions, there is a concomitant increase in both the amount of nuclear YY1 expression per cell, and in the proportion of cells with nuclear staining.

Cytoplasmic YY1 staining distributions are shown in FIGS. 5C and 5D, and their benign versus malignant expression compared in Table 2 (p<0.0001). In benign tissues, cytoplasmic staining is predominantly limited to weak expression, only 30% of normal and 18% of BPH-containing spots stained to at least a moderate cytoplasmic intensity. In contrast, 72% of tumor and 86% of PIN stained at that level. Staining frequency grouped with a cutoff of 50% positive staining is seen in FIG. 5D. Interestingly, both the benign and neoplastic tissue components stained predominantly with high frequency, or rarely, not at all. >99% of all spots had either 100% of the cells stained, or 0% (detailed graph not shown). Therefore, in the neoplastic lesions, there is an increase in the amount of cytoplasmic YY1 expression per cell, but the proportion of cells staining is commonly high, whether neoplastic or benign. Nonetheless, cytoplasmic positivity remains significantly higher in neoplastic compared to benign spots (Table 2, p<0.0001).

In benign tissues, the per spot relationship between YY1 staining intensity and positivity were highly correlated. In the nuclear and cytoplasmic compartments, the Pearson correlations were 0.82 (p<0.0001) and 0.60 (p<0.0001), respectively. In neoplastic tissues, the YY1 staining intensity and positivity were highly correlated only when looking at the nuclear compartment (Correlation=0.73, p<0.0001). In the cytoplasm, while intensities vary appreciably in tumor, the frequency remains almost exclusively high, and thus the correlation is reduced (Correlation=0.34, p<0.0001).

Table 1 presents a breakdown of the YY1 groups versus clinicopathologic parameters. Despite a per spot trend of increasing YY1 in the higher Gleason grade histologies (Han, M. et al. Urol. Clin. North Am., 28:555-65 (2001); Ghosh, J. et al. Proc. Natl. Acad. Sci. USA, 95:13182-13187 (1998); Thompson, C. B. Science, 267:1456-62 (1995)), the dichotomized YY1 patient groups were not associated with the overall case Gleason score (P=0.53). They were also not associated with pT stage (P=0.55); lymph node status (P=0.38); Group stage (P=0.27); capsular invasion (P=0.16); organ confinement (P=0.17); or the log of the highest preoperative PSA level (P=0.12). While an increase in the proportion of seminal vesicle invasion was seen in the low expressing YY1 case group, (reflected in a reduction of organ confinement and increase in pT stage), this factor did not reach statistical significance, (P=0.29). As expected, there was no difference in tumor margin status between dichotomized YY1 groups. Of note, all low expressing YY1 cases were negative for lymph nodes, as were 95% of higher expressing cases, making the cohort largely a non-metastatic, clinically localized, patient group.

### Time to Recurrence and Cox Regression

Recurrence-tree time data was available for 190 patient cases. Because of the highly correlated per case nuclear and cytoplasmic staining intensities and frequencies, we found no benefit in producing integrated staining values from these characteristics, and instead concentrated our analyses on intensity or frequency separately. Cytoplasmic expression itself, nor ratios of cytoplasmic to nuclear expression, showed any significant correlations to the outcomes data (data not shown). We found that our strongest YY1 variable was the median nuclear staining positivity of the pooled tumor spots in each case. This measure also demonstrated the highest difference between benign and neoplastic histologies (Table 2). Nonetheless, when this pooled YY1 expression value was used as a continuous variable in a univariate Cox regression model we found only weak evidence of association with recurrence time (P=0.091). However, when this variable was dichotomized as described above, we found that intact YY1 expression is protective (Table 3; P=0.038; hazard ratio 0.47; 95% CI 0.23-0.96). YY1 remained a significant predictor in low-grade patients (Gleason Score 2-6, N=112; P=0.036; hazard ratio 0.31, 95% CI 0.10-0.93). However, YY1 was not a significant predictor in high grade patients (P=0.48).

FIGS. 6A and 6B show the Kaplan Meier estimates of recurrence-free time distributions in all 190, and in the 112 low grade patients, respectively. YY1 nuclear expression leads to significantly different survival distributions in both patient groups. YY1 significantly reduced the risk of recurrence, log rank P=0.0327 and log rank P=0.0263, respectively. When all patients are considered together, we see a median recurrence-free interval of 52 months in low expressing tumors, compared to >163 months in high expressing tumors.

Only 32% of the YY1 expressing group showed tumor recurrence (68% were censored), in contrast with a 60% recurrence (40% censored cases) in the group with reduced YY1 expression. In high grade patients (N78) YY1 expression was not associated with increased recurrence risk (P=0.476).

The multivariate Cox proportional hazards model was fitted using known prognosticators; seminal vesicle status, Gleason score, capsular invasion, and the log of the highest preoperative (logPreOp) PSA. Since seminal vesicle status forms a cut-off surrogate for pathology pT3b stage, and organ confinement produces a significant confounder with capsule invasion and seminal vesicle status, stage and organ confinement were not included in the multivariate analysis. After adjusting for these known prognosticators, the significance of the association between YY1 expression and recurrence time was much reduced when all cases (Table 3; P=0.089; hazard ratio 0.51, 95% CI 0.24-1.11), or when only low grade cases were considered (P=0.067; hazard ratio 0.33, 95% CI 0.10-1.081). Similar to the univariate Cox analysis, we found no evidence for association in high grade patients (P 0.56; hazard ratio 0.72, 95% CI 0.24-2.15).

### Discussion

The transcriptional factor YY1 has been examined in many studies for functions in the regulation of gene expression. The findings that YY1 regulates the expression and sensitivity of tumor cell lines to TNF-α family-mediated signals for apoptosis suggested that it may have a role in tumor progression. Until now however, the prognostic significance of YY1 in cancer has not been examined. Significantly, we observed that for both nuclear and cytoplasmic staining, there was increased YY1 expression in tumor and PIN samples compared to matched morphologically normal or BPH tissues. Moreover, we demonstrate here for the first time, in a retrospective series, that low nuclear YY1 expression predicted increased risk of recurrent disease following prostatectomy. Thus, YY1 expression is of prognostic significance in prostate cancer.

PIN displayed the highest overall nuclear and cytoplasmic expression of YY1 protein, just above the levels of the highest expression seen in invasive tumors, which were seen in Gleason grades 3-5, with notably lower expression in low Gleason grade 1-2 tumors. This may indicate that YY1 is most active in the processes of in situ tumor formation, and in progression of low-grade tumors into those of high grade. This also may hint at a potentially epigenetic regulation of YY1. Interestingly, BPH was consistently the lowest expressing histology, both in the nuclear and cytoplasmic subcellular localizations. BPH may be thought of as a weakly proliferative lesion whose growth is necessarily well regulated through normally functioning intact apoptotic pathways. Therefore, a normal reduction in YY1 content may allow Fas-mediated apoptosis to proceed with appropriate activity in these tissues.

The present study strengthens the theory that YY1 may act as a tumor suppressor in prostate cancer, because low nuclear expression is associated with recurrence. These findings are reminiscent of a recent report by Pellikainen et al (Pellikainen, J. et al., Clin. Cancer Res., 8:3487-95 (2002)), who found that reduced nuclear expression of the transcriptional factor AP-2 correlated with aggressive breast cancer. In other analyses, down-regulation of AP-2 predicted poor survival in stage 1 cutaneous malignant melanoma (Karjalainen, J. M. et al., J. Clin. Oncol., 16:3584-91 (1998)), ovarian cancer (Anttila, M. A. et al., Br. S. Cancer, 82:1974-83 (2000)), cervical neoplasia (Hietala, K. A. et al., J. Pathol. 183:305-10 (1997)) and colorectal carcinoma (Ropponen, K. M. et al, J. Clin. Pathol., 54:533-8 (2001)). It seems that functional AP-2 protein is needed to promote cellular growth balance. The nuclear localization of YY1 may also indicate functional activity of the transcription factor since several genes are regulated by YY1. The tumor cells have a preferential YY1 transcription activity by redistributing the protein to the nucleus through the cytoplasm. For instance, in ovarian cancer, high cytoplasmic AP 2 was a favorable sign whereas nuclear expression with low cytoplasmic expression indicated risk of recurrence.

There is evidence that a large number of cancer related genes can be simply switched on or off over a relatively short period of time. This suggests the existence of control molecules that can alter the expression of a larger group of genes. YY1 is a known repressor of gene expression. When YY1 is activated a substantial number of other genes are shut down. Products of these genes may promote tumor development and their repression by YY1 therefore slows tumor progression. A study by Varambally et al (Varambally, S. et al., Nature, 419:624-9 (2002)) recently reported that a gene is activated in advance prostate cancer encoding the EZH-2 protein, another known repressor of gene expression (Laible, G. et al., EMBO J., 16:3219-32 (1997)). When the EZH-2 gene is activated in prostate cancer, a substantial number of other genes are similarly shut down. Some of the products of these genes appear to suppress tumor development, which suggests that their repression by EZH-2 could accelerate tumor progression towards metastasis. The data from Varambally and his colleagues (Varambally, S. et al., Nature, 419:624-9 (2002)) indicate the presence of EZH-2 at the time of diagnosis correlates with future tumor recurrence. This protein along with other molecules such as thymosin beta-15 (Chakravatri, A. et al., Urology, 55:635-8 (2000)) and PTEN (Maehama, T. et al., Trends Cell Biol., 9:125-8 (1999)) may offer physicians new prediction tools with which to guide decisions about how and when to treat prostate cancer patients.

Previous findings in cell culture experiments showing an inverse correlation between YY1 and Fas expression and sensitivity to FasL-induced apoptosis suggested that elevated expression of YY1 activity may correlate with poor prognosis due to the resistance of tumor cell destruction by the immune system. However, in the present study, we have found that, contrary to this hypothesis, low nuclear expression associated with an increased risk of tumor recurrence.

### Tables

TABLE 1
Relationship of YY1 nuclear expression with clinicopathologic parameters (N-190 patients)

| | | | “Low” YY1 | | “High” YY1 | | | |
|---|---|---|---|---|---|---|---|---|
| YY1 Nuclear | | | Positivity < 50% | | Positivity ≧ 50% | | | |
| Expression Frequency | All Patients | | (Percent of Total) | | (Percent of Total) | | P-Value^{a} | |
| Total Case | 190 | | 15 | (8) | 175 | (92) | | |
| Age At Surgery, Median | 65 | (range 46-76) | 62 | (range 54-75) | 65 | (range 46-76) | | |
| Gleason Score | | | | | | | 0.53 | (NS) |
| 2-6 | 112 | (59) | 10 | (67) | 102 | (58) | | |
| 7-10 | 78 | (41) | 5 | (33) | 73 | (42) | | |
| Pathology pT Stage | | | | | | | 0.55 | (NS) |
| pT2 (2a and 2b) | 128 | (67) | 8 | (53) | 120 | (69) | | |
| pT3a | 30 | (16) | 3 | (20) | 27 | (15) | | |
| pT3b | 32 | (17) | 4 | (27) | 28 | (16) | | |
| pT4 | 0 | (0) | 0 | (0) | 0 | (0) | | |
| Group Stage | | | | | | | 0.27 | (NS) |
| II | 124 | (65) | 8 | (53) | 116 | (66) | | |
| III | 57 | (30) | 7 | (47) | 50 | (29) | | |
| IV | 9 | (5) | 0 | (0) | 9 | (5) | | |
| Lymph Node Status | N = 187 | | N = 14 | | N = 173 | | 0.38 | (NS) |
| (positive cases/total | | | | | | | | |
| lymphadenectomies) | | | | | | | | |
| Positive | 9 | (5) | 0 | (0) | 9 | (5) | | |
| Negative | 178 | (95) | 14 | (100) | 164 | (95) | | |
| Organ Confined^{b} | N = 180 | | N = 13 | | N = 167 | | 0.17 | (NS) |
| Yes | 115 | (64) | 6 | (46) | 109 | (65) | | |
| No | 65 | (36) | 7 | (54) | 58 | (35) | | |
| Tumor Margins | | | | | | | 0.95 | (NS) |
| Positive | 62 | (33) | 5 | (33) NA | 57 | (33) NA | | |
| Negative | 128 | (67) | 10 | (67) NA | 118 | (67) NA | | |
| Capsular Invasion | | | | | | | 0.16 | (NS) |
| No Invasion | 44 | (23) | 0 | (0) | 44 | (25) | | |
| Invasion | 107 | (56) | 12 | (80) | 95 | (54) | | |
| Extension | 39 | (21) | 3 | (20) | 36 | (21) | | |
| Seminal Vesicle | | | | | | | 0.29 | (NS) |
| Invasion | | | | | | | | |
| No | 158 | (83) | 11 | (73) | 147 | (84) | | |
| Yes | 32 | (17) | 4 | (27) | 28 | (16) | | |
| LOG Higest PreOpSA | (range 0.3-4.3) | | (range 1.7-3.9) | | (Range 0.3-4.3) | | 0.12 | (NS) |
| (N = 169) | Median 2.2 | | Median 2.6 | | Median 2.2 | | | |
| | Average 2.3 | | Average 2.6 | | Average 2.2 | | | |
| Total Follow-up, months | (range 0-182) | | (range 9-144) | | (Range 0-182) | | 0.30 | (NS) |
| | Median 84 | | Median 98 | | Median 81 | | | |
| | Average 78 | | Average 86 | | Average 78 | | | |
| Recurrence^{c} | | | | | | | 0.029 | |
| Yes | 65 | (34) | 9 | (60) | 56 | (32) | | |
| No | 125 | (66) | 6 | (40) | 119 | (68) | | |

| | | | |
|---|---|---|---|
| Total Follow-up in | (range 4-182) | (range 47-144) | (range 4-182) |
| Recurred group (N = 65) | Median 98 | Median 98 | Median 97 |
| | Average 95 | Average 95 | Average 96 |
| Total Follow-up in | (range 0-163) | (range 9-120) | (range 0-163) |
| Non-Recurring group | Median 72 | Median 90 | Median 71 |
| (N = 125) | Average 70 | Average 72 | Average 70 |

| | |
|---|---|
| Time to Recurrence | 0.038, |
| | hazard |

| | | | | |
|---|---|---|---|---|
| (Recurred Group), | (range 1-115) | (range 2-115) | (range 1-98) | ratio 0.47, |
| months | Median 21 | Median 15 | Median 23 | (CI 0.23- |
| | Average 28 | Average 33 | Average 27 | 0.96)^{d} |

Survival

| | | | |
|---|---|---|---|
| Alive | 160 | 13 | 147 |

| | | | |
|---|---|---|---|
| Dead (All causes) | 9 | 0 (0% of total; 0% | 9 (5% of total; 32% |
| | | of dead) | of dead) |

| | | | |
|---|---|---|---|
| Time to Death in those | (range 21-143)′ | (range NA) | (range 21-143) |
| dead of disease, months | Median 68 | Median NA | Median 68 |
| | Average 75 | Average NA | Average 75 |
| Total follow-up in those | (range 2-182) | (range 9-120) | (range 2-182) |
| Alive, months (N = 160) | Median 84 | Median 98 | Median 84 |
| | Average 77 | Average 79 | Average 77 |

^{a}P-value are determined by the Kruskal-Wallis test unless otherwise specified
^{b}Organ Confined = no capsular extension and/or seminal vesicle and/or lymph node involvement
^{c}Recurrence = PSA elevation raising >0.2 ng/ml status post radical prostatectomy
^{d}P-value determined by Cox proportional hazards model

TABLE 2
Association of Benign^{a} and Neoplastic^{b} Tissue Groups
by Nuclear or Cytoplasmic YY1 Expression Variables
(per spot comparison; N = 1061)

| | Benign versus Neoplastic Expression^{c} | |
|---|---|---|
| YY1 Expression Scoring Method | Chi Square | P value |
| Nuclear Intensity | 107.5 | <0.0001 |
| Nuclear Positivity^{d} | 216.3 | <0.0001 |
| Cytoplasmic Intensity | 199.6 | <0.0001 |
| Cytoplasmmic Positivity | 34.6 | <0.0001 |

^{a}n = 333 array spots
^{b}n = 728 array spots
^{c}Kruskal-Wallis Test
^{d}variable measure used for clinical outcomes studies

TABLE 3
Cox Proportional Hazards analyses

| | | | Univariate | Multivariate |
|---|---|---|---|---|
| | Univariate (All | Multivariate (All | (Low Gleason | (Low Gleason |
| | Patients, N = 190) | Patients, N = 169) | Score^{a}, N = 112) | score^{a}, N = 102) |
| Variable | P-value; Hazard | P-value; Hazard | P-value; Hazard | P-value; Hazard |
| | Ratio; (95% | Ratio; (95% | Ratio; (95% | Ratio; (95% |
| | Confidence | Confidence | Confidence | Confidence |
| | Interval) | Interval) | Interval) | Interval) |
| Seminal vesicle | <0.0001 | 0.0024 | 0.0043 | 0.020 |
| invasion (Stage ≧ | 4.61 | 2.60 | 6.085 | 4.85 |
| pT3b) | (2.73-7.76) | (1.40-4.83) | (1.76-21.04) | (1.28-18.43) |
| Gleason > 7 | <0.0001 | 0.0052 | NA | NA |
| | 3.96 | 2.59 | | |
| | (2.35-6.67) | (1.33-5.047) | | |
| Log (preoperative | 0.0001 | 0.10 | 0.22 | 0.025 |
| PSA) | 1.88 | 1.34 | 2.15 | 2.33 |
| | (1.36-2.59)^{b} | (0.94-1.91) | (1.12-4.13)^{c} | (1.11-4.89) |
| Capsular Invasion | 0.0015 | 0.014 | 0.0067 | 0.0060 |
| | 1.82 | 1.78 | 2.41 | 3.40 |
| | (1.26-2.64) | (1.13-2.82) | (1.28-4.56) | (1.42-8.13) |
| YY1 nuclear | 0.038 | 0.089 | 0.036 | 0.067 |
| positivity | 0.47 | 0.51 | 0.31 | 0.33 |
| (dichotomized)^{d} | (0.23-0.96) | (0.24-1.11) | (0.10-0.93) | (0.10-1.081) |

^{a}Gleason Score 2-6
^{b}N-169
^{c}N = 102
^{d}YY1 < 50% (n = 15); YY1 > 50% (n = 175) positive

### Example 2

### Overexpression of YY1 in Multiple Myeloma Cancer Cells

This study investigated the expression of YY1 in multiple myeloma (MM) with the objective of determining whether YY1 plays a role in the progression and drug-resistance of MM. We have initiated these studies by examining nine bone marrow (BM) samples derived from patients with MM. Immunohistochemical studies were performed for the detection and cytoplasmic or nuclear localization of YY1 in the MM cells and also in adjacent normal mature and immature cells. The intensity of staining by the anti-YY1 antibody was scored and the relative intensity was calculated. Two slides from each patient were analyzed and 200 cells per slide were scored. Mean intensities of all samples were calculated and the data were subjected to statistical analysis. YY1 expression in normal BM was low and primarily of cytoplasmic origin. In contrast, YY1 was significantly overexpressed in MM cells. The mean intensity in the MM was approximately three-four fold higher than that of the normal cells and was primarily of nuclear origin (p-value <0.05). The signals that control shuttling YY1 are undefined. The expression of YY1 in normal mature and immature cells was low and there was comparable staining in the cytoplasm and the nucleus. Analysis of the cell distribution expressing YY1 by flow cytometry revealed that greater than 50% of the cells in the CD38+ subset expressed YY1. In addition, the MM tumor cells also expressed high level of pleiotrophin (PTN) and the patients had high levels of circulating PTN. PTN is a growth factor for MM and PTN transcription is regulated by an initiator element and could thus be a target of YY1-mediated transcriptional control. These findings suggest that YY1 overexpression is involved in the pathogenesis of MM and is a target for therapeutic intervention.

### Example 3

### Overexpression of YY1 in Lymphoma Cancer Cells

We have shown that overexpression of YY1 regulates the resistance of tumor cells to TRAIL-induced apoptosis (Ng and Bonavida, 2002, Molecular Cancer Therapeutics 1:1051-1058, Huerta-Yepez, et al., 2004, Oncogene 23:4993-5003). One mechanism of AIDS-NHL immune escape may be due to overexpression of YY1. Tissue arrays containing formalin fixed, paraffin embedded sections from AIDS lymphoma were obtained from the AIDS and Cancer Specimen Resource (ACSR) of the National Cancer Institute (NCI. These arrays consisted of 21 Burkitt, 29 Large Cell Lymphoma, and 6 Small Cell Lymphoma. Immunohistochemistry was performed for the expression of YY1. The arrays were scored and both the percent of positive cells and the intensity were recorded and the data were analyzed statistically. The findings reveal that YY1 was overexpressed in the majority of the AIDS-NHL patient specimens. In addition, there was a significant correlation between YY1 expression in all 3 types of lymphoma. YY1 is a marker for tumor unresponsiveness to immune-mediated cytotoxic therapies. Furthermore, inhibitors of YY1 expression/activity are targets for therapeutic intervention when combined with immunotherapy.

### Example 4

### Mechanisms of Transcriptional Upregulation of DR5 by Chemotherapeutic Drugs and Sensitization to Trail-Induced Apoptosis

TRAIL, a member of the TNF family, has been shown to kill sensitive tumor cells with minimal toxicity to normal tissues and is a new candidate for immunotherapy in the treatment of drug-refractory tumor cells. However, many drug-resistant tumor cells are also resistant to TRAIL and such tumors require sensitization to reverse TRAIL resistance. We, and others, have reported that several sensitizing agents (ex. Act.D, CDDP, ADR, chemical inhibitors, etc.) in combination with TRAIL result in significant apoptosis and synergy is achieved. In addition, several sensitizing agents resulted in the upregulation of DR5 expression and whose mechanism is not known (Ng et al., Prostate, 53: 286, 2002; Huerta-Yepez et al., Oncogene, 23: 4993, 2004). We hypothesize that the sensitizing drugs may, directly or indirectly, interfere with a transcription repressor factor. Examination of the DR5 promoter revealed the presence of one binding site for the transcription repressor Yin Yang 1 (YY1) and that YY1 may negatively regulate DR5 transcription. This hypothesis was tested by examining a luciferase reporter system (pDR5 wild type) and plasmids in which the YY1-binding site was either deleted (pDR5/-605) and/or mutated (pDR5-YY1 mutant). Using the PC3 prostate tumor cell line as a model system, we demonstrate that PC3 transfected with pDR5 resulted in basal luciferase activity and treatment with CDDP or ADR significantly augmented luciferase activity. PC3 cells transfected with pDR5/-605 or pDR5-YY1 also resulted in significant potentiation of the basal luciferase activity. These findings demonstrate that YY1 negatively regulates DR5 transcription and regulates tumor cells' resistance to TRAIL. Inhibition of YY1 sensitized tumor cells to TRAIL-induced apoptosis. The present findings demonstrate that drugs-induced upregulation of DR5 expression is mediated via inhibition of the transcription repressor YY1. The findings show that tumor cells overexpressing YY1 will be resistant to TRAIL-induced apoptosis. Therefore, inhibition of YY1 is clinically useful in the therapeutic application of TRAIL in resistant tumor cells.

### Example 5

### Regulation of the TRAIL Receptor DR5 Expression by the Transcription Repressor Yin Yang 1 (YY1)

TRAIL is a member of the TNF-α superfamily and has been shown to be selectively cytotoxic to sensitive tumor cells. However, most tumors are resistant to TRAIL and need to be sensitized to undergo apoptosis. We have recently reported that TRAIL-resistant human prostate carcinoma (CaP) cell lines can be sensitized by various NF-κB inhibitors (e.g. NO donor DETANONOate) (Huerta-Yepez et al., Oncogene, 23: 4993, 2004), and sensitization correlated with upregulation of DR5 expression. We hypothesized that a gene product(s) regulated by NF-κB with DR5 repressor activity may be responsible for the DR5 regulation. The transcription repressor Yin-Yang 1 (YY1), under the regulation of NF-κB (NF-κB has a putative DNA-binding site (−804 to −794 bp)), was investigated. Treatment of CaP PC-3 cells with DETANONOate resulted in significant upregulation of DR5 expression as determined by flow, RT-PCR and western. Further, treatment of PC-3 cells with DETANONOate inhibited both NF-κB and YY1 DNA-binding activity and DETANONOate also inhibited YY1 expression. Treatment of PC-3 cells with YY1 siRNA resulted in upregulation of DR5 expression and sensitization to TRAIL-induced apoptosis. To examine directly the role of YY1 in the regulation of DR5 expression, a DR5 luciferase reporter system (pDR5) was used. Two constructs were generated, namely the pDR5/-605 construct with a deletion in the promoter region containing the putative YY1 DNA-binding region (−1224 to −605) and a construct pDR5-YY1 with a mutation of the YY1 DNA-binding site. Transfection of PC-3 cells with these two constructs resulted in significant (3-fold) augmentation of luciferase activity over baseline suggesting the repressor activity of YY1. Altogether, the present findings demonstrate that NF-κB-mediated downregulation of DR5 expression is, achieved in part, through the transcription repressor YY1 that negatively regulates DR5 transcription and expression and hence, YY1 regulates resistance to TRAIL-induced apoptosis. Thus, inhibition of either NF-κB or YY1 results in the upregulation of DR5 expression and sensitization of tumor cells to TRAIL-induced apoptosis. These findings show that inhibitors of YY1 expression and/or activity may be useful in the treatment of TRAIL-resistant tumor cells when used in combination with TRAIL or TRAIL agonist antibodies.

### Example 6

### Regulation of Chemoresistance and Immune Resistance OF B-NHL Cell Lines by Overexpression of YY1 and Bcl-X1, Respectively: Reversal of Resistance by Rituximab

We have recently reported that treatment of B-Non-Hodgkin's Lymphoma (NHL) cell lines with rituximab (anti-CD20 antibody) sensitizes the tumor cells to both chemotherapy and Fas-induced apoptosis (Jazirehi and Bonavida, 2005, Oncogene, 24:2121-2145). This study investigated the underlying molecular mechanism of rituximab-mediated reversal of resistance. Treatment of B-NHL cell lines inhibited the constitutively activated NF-κB. Cells expressing dominant active IκB or treated with NF-κB specific inhibitors were sensitized to both drugs and FasL agonist mAb (CH-11)-induced apoptosis. Downregulation of Bcl-xL expression via inhibition of NF-κB activity correlated with chemosensitivity. The direct role of Bcl-xL in chemoresistance was demonstrated by the use of Bcl-xL overexpressing Ramos cells, Ramos HA-BclxL (gift from Genhong Cheng, UCLA), which were not sensitized by rituximab to drug-induced apoptosis. However, inhibition of Bcl-xL in Ramos HA-Bcl-x resulted in sensitization to drug-induced apoptosis. The role of Bcl-xL expression in the regulation of Fas resistance was not apparent as Ramos HA-Bcl cells were as sensitive as the wild type cells to CH-11-induced apoptosis. Several lines of evidence support the direct role of the transcription repressor Yin-Yang 1 (YY1) in the regulation of resistance to CH-11-induced apoptosis. Inhibition of YY1 activity by either rituximab, the NO donor DETANONOate, or following transfection with YY1 siRNA all resulted in upregulation of Fas expression and sensitization to CH-11-induced apoptosis. These findings show two complementary mechanisms underlying the chemo-sensitization and immuno-sensitization of B NHL cells by rituximab via inhibition of NF-κB. The regulation of chemoresistance by NF-B is mediated via Bcl-xL expression whereas the regulation of Fas resistance by NF-B is mediated via YY1 expression and activity. These findings show that drug-resistant NHL tumor cells is sensitive to immune-mediated therapeutics.

### Example 7

### Chemosensitization of Drug-Resistant Ramos B-NHL to Drug-Induced Apoptosis: YY1 Expression is Decreased in Response to Cytoskeletal-Interacting Drugs

The transcription factor Yin Yang 1 (YY1) regulates cellular differentiation, hematopoiesis, response to apoptotic stimuli, pathogenesis of cancer and its increased expression is associated with inhibition of differentiation of progenitor cells. We and others have previously shown that expression levels of YY1 also correlate with drug sensitivity in cancer cells. A comparison between the wild type (wt) Ramos, with the recently generated rituximab-resistant Ramos (Ramos R R) clones, revealed that, unlike wt Ramos, Ramos RR1 cannot be chemosensitized by rituximab and exhibited higher drug resistance. Further, there was enhanced YY1 expression in Ramos RR1. We hypothesized that overexpression of YY1 may be, in part, responsible for drug-resistance in Ramos RR1 and its inhibition can reverse resistance. This study investigated whether the heightened expression of YY1 in Ramos RR1 cells can be reversed by a panel of drugs used in combination. Ramos and Ramos RR1 cells were treated with vincristine, VP-16, CDDP, and ADR, and the NF-κB inhibitors Bortezomib and DHMEQ. Treatment of Ramos RR1 with the NF-B inhibitor, in combination with any of the above chemotherapeutic drugs, reversed the acquired drug-resistance and synergy was achieved. Noteworthy, in Ramos RR1 cells, only vincristine (or in combination with NF-κB inhibitors) significantly abrogated or diminished YY1 expression. Similarly, in the prostatic cell line PC-3, 2-methoxyestradiol, another cytoskeletal interacting drug, resulted in marked reduction of YY1 expression level and activity (assessed by a luciferase reporter assay). These results show that YY1 overexpression may regulate the resistance of B-NHL to a selected group of drugs but not all drugs. The studies also show that agents that modulate YY1 expression/activity are useful therapeutics when used in combination with chemotherapeutic drugs in the treatment of drug and rituximab-resistant B-NHL.

### Example 8

### Nitric Oxide Decreases the Transcription Repressor Activity of Yin-Yang 1 (YY1) via S-Nitrosylation: Role in the Immunosensitization of Tumor Cells to Apoptosis

Yin-Yang 1 (YY1) is a transcription factor that may activate or repress gene expression. We have reported the upregulation of the TNF receptor family members by nitric oxide (NO) resulting in the sensitization of tumor cells (e.g. ovarian, renal, lung, prostate, lymphoma) to TNF family-mediated apoptosis. The sensitization by NO was suggested to be mediated in part to S-nitrosylation of the transcription repressor YY1 and consequently, the inhibition of its DNA-binding activity in the silencer region of the receptor promoter. In this study, we examined the direct S-nitrosylation of YY1 using the human prostate cancer cell line, PC-3, as model. Culture cells were incubated for 18 h in the presence of various concentrations of the NO donor DETANONOate (500 μM, 1000 μM) that sensitized PC-3 to Fas ligand- and TRAIL-mediated apoptosis. Subsequently, we analyzed for S-nitrosylation of YY1 by various methods. Using immunohistochemistry, we found that general S-nitrosylation of proteins was increased after treatment with NO. Noteworthy, significant constitutive S-nitrosylated proteins were detected. Further, using double immunofluorescence staining microscopy, we co-localized the presence of S-nitrosylated proteins and YY1. In addition, a specific significant increase in YY1-SNO protein was determined in culture cells exposed to NO by immunoprecipitation with anti-Cys SNO antibody followed by immunodetection of YY1. These findings were corroborated by demonstrating that immunoprecipitation of NO-treated PC-3 cells by anti-SNO antibody revealed that YY1 was S-nitrosylated using the method by Miles et al (Meth Enzym., 268:105, 1996). These, findings altogether reveal a novel mechanism of YY1 regulation by nitric oxide showing direct S-nitrosylation of this transcription repressor, the consequent decrease in DNA-binding activity and derepression of gene transcription.

### Example 9

### Rituximab-Mediated Inhibition of the Transcription Repressor Yin-Yang 1 (YY1) in NHL B Cell Lines: Upregulation of Fas Expression and Sensitization to Fas-Induced Apoptosis

We have reported that rituximab triggers and inhibits anti-apoptotic gene products in NHL B-cell lines resulting in sensitization to drug-induced apoptosis (Alas et al., Clin. Cancer Res. 8:836, 2001; Jazirehi et al., Mol. Cancer. Therapy 2:1183, 2003; Vega et al., Oncogene 23:3530, 2004). This study investigated whether rituximab also modifies intracellular signaling pathways resulting in the sensitization of NHL cells to Fas-induced apoptosis. Treatment of the NHL cell lines (2F7, Ramos, and Raji) with rituximab (20 μg/ml) sensitized the cells to CH-11 (FasL agonist mAb)-induced apoptosis and synergy was achieved. Fas expression was up-regulated by rituximab as early as 6 h post treatment as determined by flow cytometry, RT-PCR, and Western. Rituximab inhibited both the expression and activity of the transcription repressor Yin-Yang 1 (YY1) that negatively regulates Fas transcription. Inhibition of YY1 resulted in upregulation of Fas expression and sensitization of the tumor cells to CH-11-induced apoptosis. Downregulation of YY1 expression was the result of rituximab-induced inhibition of both the p38MAPK signaling pathway and constitutive NF-κB activity. The dual roles of NF-κB and YY1 in the regulation of Fas expression were corroborated by the use of a dominant-active inhibitor of NF-κB (Ramos IκB-ER mutant) and YY1 siRNA, respectively. The role of rituximab-mediated inhibition of the p38MAPK/NF-κB/YY1 pathways, which result in both Fas upregulation and sensitization to CH11-induced apoptosis, was corroborated by the use of specific chemical inhibitors directed at various targets of these pathways. Rituximab-mediated sensitization to CH-11-induced apoptosis was executed through the Type II mitochondrial apoptotic pathway. Altogether, these findings provide a novel mechanism of rituximab-mediated signaling by inhibiting the p38MAPK/NF-κB/YY1 pathways and resulting in the sensitization of B NHL to Fas-induced apoptosis. These findings show an additional mechanism of rituximab-mediated effect in vivo in addition to complement-dependent cytotoxicity (CDC) and antibody-dependent cellular cytotoxicity (ADCC).

The following references provide information relevant to the present invention.
Baritaki S, Neshat M, Huerta-Yepez S, Murdock B, Sakai T, Spandidos D A, Bonavida B. Mechanisms of transcriptional upregulation of DR5 by chemotherapeutic drugs and sensitization to TRAIL-induced apoptosis. Proceedings of the AACR, Volume 46, 2005.
Sara Huerta-Yepez, Mario Vega, Saul E. Escoto-Chavez, Benjamin Murdock, Hermes Garban, Toshiyuki Sakai and Benjamin Bonavida. Regulation of the TRAIL receptor DR5 expression by the transcription repressor Yin Yang 1 (YY1). Proceedings of the AACR, Volume 46, 2005.
Sara Huerta-Yepez, Ph.D., Stravoula Baritaki, Ph.D., Angeles Hernandez-Cuecto, M.D., Yu-Mei Anguino-Hernandez, Mehran Neshat, Ph.D., Haiming Chen, M.D., Richard A. Campbell, Melinda S. Gordon, Ph.D., James R. Berenson, Ph.D. and Benjamin Bonavida, Ph.D. Overexpression and preferential nuclear translocation of the transcription factor Yin Yang 1 (YY1) in human bone marrow-derived multiple myeloma. 2005. ASH Abstract.
Mario I. Vega, Ph.D., Ali R. Jazirehi, Ph.D., Sara Huerta-Yepez, Ph.D. and Benjamin Bonavida, Ph.D. Regulation of chemoresistance and immune resistance of B-NHL cell lines by overexpression of YY1 and Bcl-xl, respectively: reversal of resistance by rituximab. 2005. ASH Abstract.
Sara Huerta-Yepez, Ph.D., Mario Vega, Ph.D., Dorina Gui, M.D., Jonathan Said, M.D.3 and Benjamin Bonavida, Ph.D.Analysis of YY1 and XIAP expression, proteins that regulate resistance, in AIDS-NHL tissue arrays. 2005. ASH Abstract.
Mehran Neshat, Ph.D.1 and Benjamin Bonavida, Ph.D., Chemosensitization of drug-resistant Ramos B-NHL to drug-induced apoptosis: YY1 expression is decreased in response to cytoskeletal-interacting drugs. 2005. ASH Abstract.
David B. Seligson, Lee Goodglick, Steve Horvath, Sara Huerta-Yepez, Stephanie Hanna, Hermes Garban, Alice Roberts, Tao Shi, David Chia, Benjamin Bonavida. Diagnostic and prognostic significance of the Ying-Yang 1 Transcription factor in human prostate cancer, Proceedings of the AACR, Volume 45, March 2004. AACR Abstract #1070
Fumiya Hongo, Hermes Garban, Sara Huerta-Yepez, Mario Vega, Ali Jazirehi, Yoichi Mizutani, Benjamin Bonavida. Nitric oxide decreases the transcription repressor activity of Yin-Yang 1 (YY1) via S-nitrosylation: Role in the immunosensitization of tumor cells to apoptosis, Proceedings of the AACR, Volume 45, 2004. ASH Abstract #4356
Mario I. Vega, Sara Huerta-Yepez, Ali R. Jazirehi, Hermes Garban, Benjamin Bonavida. Rituximab-Mediated Inhibition of the Transcription Repressor Yin-Yang 1 (YY1) in NHL B Cell Lines: Upregulation of Fas Expression and Sensitization to Fas-Induced Apoptosis. Blood, 104, 2004. ASH Abstract #287
Huerta-Yepez, S., Vega, M., Garban, H. and Bonavida B. Involvement of the TNF-a autocrine/paracrine loop, via NF-κB and YY1, in the regulation of tumor cell resistance to Fas-induced apoptosis. (submitted)
Seligson, D., Huerta, S., Horvath, S., Hanna, S., Shi, T., Gabarn, H., Chia, D., Goodglick, L. and Bonavida. B. Expression of transcription factor Yin Yang 1 in prostate cancer. Int J Oncol. 2005 27:131-41
Vega, M., Huerta-Yepez, S., Jazirehi, A. R., Garban, H. and Bonavida, B. Rituximab (chimeric anti-CD20) upregulates Fas expression in NHL B cell lines via inhibition of constitutive NF-κB and Yin Yang 1 (YY1) activities: sensitization to Fas-induced apoptosis. Oncogene. Aug. 15, 2005, pages 1-14.
Vega, M. I., Huerta-Yepez, S., Jazirehi, A. and Bonavida, B. Rituximab-Induced Inhibition of YY1 and Bcl-xL Expression in Ramos Non-Hodgkin's Lymphoma Cell Line via Ihibition of NF-κB Activity: Role of YY1 and Bcl-xL in Fas Resistance and Chemoresistance, Respectively. The Journal of Immunology, 2005, 175: 2174-2183.
Hongo, F., Huerta-Yepez, S., Vega, M., Garban, H., Jazirehi, A., Mizutani, Y., Miki, T. and Bonavida, B. Nitroysylation of the transcription repressor Yin-Yang 1 (YY1) mediates upregulation of Fas expression in cancer cells: nitric oxide (NO)-induced sensitization to Fas-mediated apoptosis. BBRC (2005) 336:692-701.
Sherilyn Gordon, Gina Akopyan, Hermes Garban and Benjamin Bonavida. Transcription Factor YY1: Structure, Function, and Therapeutic Implications in Cancer Biology. Oncogene. In Press.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of diagnosing a cancer that overexpresses YY1 protein and/or augments YY1 transcriptional activity, the method comprising the steps of:
(a) contacting a tissue sample with an antibody that specifically binds to YY1 protein; and
(b) determining whether or not YY1 protein is overexpressed in the sample; thereby diagnosing the cancer that overexpresses YY1.

2. The method of claim 1, wherein the cancer that overexpresses YY1 is selected from the group consisting of prostate cancer, ovarian cancer, renal cancer, breast cancer, colon cancer, lung cancer, leukemia, non-Hodgkin's lymphoma, multiple myeloma and hepatocarcinoma.

3. The method of claim 1, wherein the tissue sample is a needle biopsy, a surgical biopsy or a bone marrow biopsy.

4. The method of claim 3, wherein the tissue sample is at least one of fixed or embedded in paraffin.

5. The method of claim 1, wherein the antibody is a monoclonal antibody.

6. A method of diagnosing a cancer that overexpresses YY1, the method comprising the steps of:
(a) contacting a tissue sample with a primer set of a first oligonucleotide and a second oligonucleotide that each specifically hybridize to YY1 nucleic acid;
(b) amplifying YY1 nucleic acid in the sample; and
(c) determining whether or not YY1 nucleic acid is overexpressed in the sample; thereby diagnosing the cancer that overexpresses YY1.

7. The method of claim 6, wherein the cancer that overexpresses YY1 is selected from the group consisting of prostate cancer, ovarian cancer, renal cancer, breast cancer, colon cancer, lung cancer, leukemia, non-Hodgkin's lymphoma, multiple myeloma and hepatocarcinoma.

8. The method of claim 4, wherein the tissue sample is microlasar microdissected cells from a needle biopsy, a surgical biopsy, or a bone marrow biopsy.

9. The method of claim 4, wherein the first oligonucleotide comprises SEQ ID NO:1 and the second oligonucleotide comprises SEQ ID NO:2.

10. A method of providing a prognosis for a cancer that overexpresses YY1 protein or biological activity, the method comprising the steps of:
(a) contacting a tissue sample with an antibody that specifically binds to YY1 protein; and
(b) determining whether or not YY1 protein is overexpressed in the sample; thereby providing a prognosis for the cancer that overexpresses YY1.

11. The method of claim 10, wherein the cancer that overexpresses YY1 is selected from the group consisting of prostate cancer, ovarian cancer, renal cancer, breast cancer, colon cancer, lung cancer, leukemia, non-Hodgkin's lymphoma, multiple myeloma and hepatocarcinoma.

12. The method of claim 10, wherein the tissue sample is a needle biopsy, a surgical biopsy or a bone marrow biopsy.

13. The method of claim 10, wherein the antibody is a monoclonal antibody.

14. The method of claim 10, wherein the tissue sample is a metastatic cancer tissue sample.

15. The method of claim 10, wherein the tissue sample is from prostate, ovary, bone, lymph node, liver, or kidney.

16. A method of providing a prognosis for a cancer that overexpresses YY1, the method comprising the steps of:
(a) contacting a tissue sample with a primer set of a first oligonucleotide and a second oligonucleotide that each specifically hybridize to YY1 nucleic acid;
(b) amplifying YY1 nucleic acid in the sample; and
(c) determining whether or not YY1 nucleic acid is overexpressed in the sample; thereby providing a prognosis for the cancer that overexpresses YY1.

17. The method of claim 16, wherein the cancer that overexpresses YY1 is selected from the group consisting of prostate cancer, ovarian cancer, renal cancer, lung cancer, breast cancer, colon cancer, leukemia, non-Hodgkin's lymphoma, multiple myeloma and hepatocarcinoma.

18. The method of claim 16, wherein the first oligonucleotide comprises SEQ ID NO:1 and the second oligonucleotide comprises SEQ ID NO:2.

19. The method of claim 16, wherein the tissue sample is a needle biopsy, a surgical biopsy or a bone marrow biopsy.

20. The method of claim 16, wherein the tissue sample is a metastatic cancer tissue sample.

21. The method of claim 16, wherein the tissue sample is from prostate, ovary, bone, lymph node, liver, or kidney.

22. An isolated primer set, the primer set comprising a first oligonucleotide and a second oligonucleotide, the oligonucleotides comprising a nucleotide sequence of 50 nucleotides or less; wherein the first oligonucleotide comprises SEQ ID NO:1 and the second oligonucleotide comprises SEQ ID NO:2.

23. A method of localizing a cancer that overexpresses YY1 in vivo, the method comprising the step of imaging in a subject a cell overexpressing YY1 polypeptide, thereby localizing cancer in vivo.

24. The method of claim 23, wherein the cancer that overexpresses YY1 is selected from the group consisting of prostate cancer, ovarian cancer, renal cancer, breast cancer, lung cancer, colon cancer, leukemia, non-Hodgkin's lymphoma, multiple myeloma and hepatocarcinoma.

25. A method of identifying a compound that inhibits a cancer that overexpresses YY1, the method comprising the steps of:
(a) contacting a cell expressing YY1 polypeptide with a compound; and
(b) determining the effect of the compound on the YY1 polypeptide; thereby identifying a compound that inhibits the cancer that overexpresses YY1.

26. The method of claim 25, wherein the compound inhibits the binding of YY1 to a DNA sequence.

27. The method of claim 25, wherein the cell comprises a promoter sequence bound by YY1 operably linked to a reporter nucleic acid sequence.

28. The method of claim 25, wherein the cancer that overexpresses YY1 is selected from the group consisting of prostate cancer, ovarian cancer, renal cancer, lung cancer, breast cancer, colon cancer, leukemia, non-Hodgkin's lymphoma, multiple myeloma and hepatocarcinoma.

29. A method of identifying a compound that inhibits a therapy resistant cancer, the method comprising the steps of:
(a) contacting a cell expressing YY1 polypeptide with a compound; and
(b) determining the effect of the compound on the YY1 polypeptide; thereby identifying a compound that inhibits the therapy resistant cancer.

30. The method of claim 29, wherein the compound inhibits the binding of YY1 to a DNA sequence.

31. The method of claim 29, wherein the compound sensitizes the cell to apoptosis induced by cell signaling through a death receptor.

32. The method of claim 29, wherein the cancer that overexpresses YY1 is selected from the group consisting of prostate cancer, ovarian cancer, renal cancer, lung cancer, breast cancer, colon cancer, leukemia, non-Hodgkin's lymphoma, multiple myeloma and hepatocarcinoma.

33. A method of treating or inhibiting a cancer that overexpresses YY1 in a subject comprising administering to the subject a therapeutically effective amount of one or more YY1 inhibitors.

34. The method of claim 33, wherein the cancer that overexpresses YY1 is selected from the group consisting of prostate cancer, ovarian cancer, renal cancer, lung cancer, breast cancer, colon cancer, leukemia, non-Hodgkin's lymphoma, multiple myeloma and hepatocarcinoma.

35. The method of claim 33, wherein the YY1 inhibitor is an NO donor.

36. The method of claim 35, wherein the NO donor is selected from the group consisting of L-arginine, amyl nitrite, isoamyl nitrite, nitroglycerin, isosorbide dinitrate, isosorbide-2-mononitrate, isosorbide-5-mononitrate, erythrityl tetranitrate, pentaerythritol tetranitrate, sodium nitroprusside, 3-morpholinosydnonimine, molsidomine, N-hydroxyl-L-arginine, S,S-dinitrosodthiol, ethylene glycol dinitrate, isopropyl nitrate, glyceryl-1-mononitrate, glyceryl-1,2-dinitrate, glyceryl-1,3-dinitrate, glyceryl trinitrate, butane-1,2,4-triol trinitrate, N,O-diacetyl-N-hydroxy-4-chlorobenzenesulfonamide, N^{G}-hydroxy-L-arginine, hydroxyguanidine sulfate, (±)-S-nitroso-N-acetylpenicillamine, S-nitrosoglutathione, (±)-(E)-ethyl-2-[(E)-hydroxyimino]-5-nitro-3-hexeneamide (FK409), (±)-N-[(E)-4-ethyl-3-[(Z)-hydroxyimino]-5-nitro-3-hexen-1-yl]-3-pyridinecarboxamide (FR144420), 4-hydroxymethyl-3-furoxancarboxamide, (Z)-1-[2-(2-Aminoethyl)-N-(2-ammonioethyl)amino]diazen-1-ium-1,2-diolate; NOC-18; 3,3-bis(aminoethyl)-1-hydroxy-2-oxo-*1-triazene (DETA/NONOate), NO gas, and mixtures thereof.

37. The method of claim 33, wherein the YY1 inhibitor is an inhibitory RNA.

38. The method of claim 33, wherein the YY1 inhibitor is an antimitotic drug.

39. The method of claim 38, wherein the antimitotic drug is selected from the group consisting of vinca alkaloids and taxanes.

40. A method of treating or inhibiting a therapy resistant cancer in a subject comprising administering to the subject a therapeutically effective amount of one or more YY1 inhibitors.

41. The method of claim 40, wherein the one or more YY1 inhibitors are administered concurrently with another cancer therapy.

42. The method of claim 40, wherein the cancer that overexpresses YY1 is selected from the group consisting of prostate cancer, ovarian cancer, renal cancer, lung cancer, breast cancer, colon cancer, leukemia, non-Hodgkin's lymphoma, multiple myeloma and hepatocarcinoma.

43. The method of claim 40, wherein the YY1 inhibitor is an NO donor.

44. The method of claim 43, wherein the NO donor is selected from the group consisting of L-arginine, amyl nitrite, iso amyl nitrite, nitroglycerin, isosorbide dinitrate, isosorbide-2-mononitrate, isosorbide-5-mononitrate, erythrityl tetranitrate, pentaerythritol tetranitrate, sodium nitroprusside, 3-morpholinosydnonimine, molsidomine, N-hydroxyl-L-arginine, S,S-dinitrosodthiol, ethylene glycol dinitrate, isopropyl nitrate, glyceryl-1-mononitrate, glyceryl-1,2-dinitrate, glyceryl-1,3-dinitrate, glyceryl trinitrate, butane-1,2,4-triol trinitrate, N,O-diacetyl-N-hydroxy-4-chlorobenzenesulfonamide, N^{G}-hydroxy-L-arginine, hydroxyguanidine sulfate, (±)-S-nitroso-N-acetylpenicillamine, S-nitrosoglutathione, (±)-(E)-ethyl-2-[(E)-hydroxyimino]-5-nitro-3-hexeneamide (FK409), (±)-N-[(E)-4-ethyl-3-[(Z)-hydroxyimino]-5-nitro-3-hexen-1-yl]-3-pyridinecarboxamide (FR144420), 4-hydroxymethyl-3-furoxancarboxamide, (Z)-1-[2-(2-Aminoethyl)-N-(2-ammonioethyl)amino]diazen-1-ium-1,2-diolate; NOC-18; 3,3-bis(aminoethyl)-1-hydroxy-2-oxo-*1-triazene (DETA/NONOate), NO gas, and mixtures thereof.

45. The method of claim 40, wherein the YY1 inhibitor is an inhibitory RNA.

46. The method of claim 40, wherein the YY1 inhibitor is an antimitotic drug.

47. The method of claim 46, wherein the antimitotic drug is selected from the group consisting of vinca alkaloids and taxanes.


SEQUENCE LISTING (2 sequences)

SEQ ID NO: 1 - DNA, 20 bp, Artificial Sequence
  Description of Artificial Sequencefirst oligonucleotide RT-PCR amplification forward primer to detect transcription factor Yin-Yang 1 (YY1)
ggccaccaccaccaccacca

SEQ ID NO: 2 - DNA, 20 bp, Artificial Sequence
  Description of Artificial Sequencefirst oligonucleotide RT-PCR amplification reverse primer to detect transcription factor Yin-Yang 1 (YY1)
ttcttgttgcccgggtcggc